(19) United States
(12) Patent Application Publication
(10) Pub. No.: US 2011/0166136 A1
(43) Pub. Date: Jul. 7, 2011

(54) Calcium Ion Channel Modulators & Uses Thereof

(75) Inventors: Nawaz Mohammed Khan, Cambridge (GB); Svenja Burckhardt, Cambridge (GB); Julie Elaine Cansfield, Cambridge (GB); Ngoc-Tri Vo, Cambridge (GB); Richard Edward Armer, Cambridge (GB); Raymond John Boffey, Cambridge (GB)

(73) Assignee: LECTUS THERAPEUTICS LIMITED, Cambridge, Cambridgeshire (GB)

(21) Appl. No.: 13/002,374

(22) PCT Filed: Jul. 3, 2009
(86) PCT No.: PCT/GB2009/050787

§ 371 Date: Feb. 22, 2011

(30) Foreign Application Priority Data
Jul. 3, 2008 (GB) 0812192.3

(51) Int. Cl. A61K 31/5377; C07D 403/06; A61K 31/496; C07D 413/06; C07D 209/42; A61K 31/404; A61P 29/00; A61P 9/00; A61P 25/22; A61P 13/00; A61P 25/06; A61P 1/00; A61P 3/00

(52) U.S. Cl. 514/235.2; 544/373; 514/254.09; 544/144; 548/492; 514/419

(57) ABSTRACT

Compounds of formula (1), salts and pro-drugs wherein:

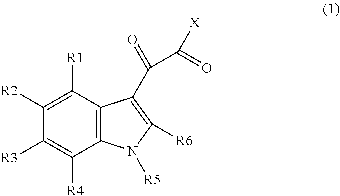

R1, R2, R3 and R4 are hydrogen, alkyl, hydroxyalkyl, halogen, haloalkyl, alkoxy, haloalkoxy, alkoxycarbonyl, carboxyl, hydroxyl, nitro, amino, monalkylamino, dialkylamino, acylamino, alkoxycarbonylamino, alkylsulphonyl, arylsulphonyl, alkylsulphonylamino, arylsulphonylamino, aminosulphonyl or cyano, or
any two of R1 to R4 that are adjacent on the ring may together represent the moiety —O—(CH₂)ₙ—O— wherein n is 1 to 3;
R5 is hydrogen or alkyl;
R6 is hydrogen or alkyl; and
X is selected from the group consisting of:
(a) groups of formula OR7 wherein R7 is hydrogen or alkyl which is optionally substituted with a substituent selected from alkylsulfonylalkyl, saturated or partially unsaturated heterocyclic, alkoxy, carboxyl, nitro, amino, monalkylamino, dialkylamino, halogen, and alkoxycarbonyl,
provided that when R7 is hydrogen or ethyl, then R1, R2, R3 and R4 cannot be selected from hydrogen, halogen and alkyl; and
(b) groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms, said saturated or partially unsaturated heterocyclic group optionally further being substituted by one or more substituents selected from alkyl, halogen, haloalkyl, alkoxy, alkoxycarbonyl, carboxyl, nitro, amino, monalkylamino, dialkylamino and hydroxyl,
provided that:
(i) when R8+R9+N=piperazine, and ≧1 of R1 to R4 are hydrogen, hydroxyl, nitro, amino, alkylamino, dialkylamino, alkoxycarbonylamino, halogen, alkoxy or alkyl, the nitrogen atom at the 4-position of the piperazine is not alkyl substituted,
(ii) when each of R1, R2, R3, R4, R5 and R6 is hydrogen, X is not unsubstituted piperazinyl or unsubstituted morpholino,
(iii) when each of R1, R2, R4, R5 and R6 is hydrogen and R3 hydrogen, bromine or hydroxyl, X is not methoxy,
(iv) when each of R2 and R3 is methoxy or they together represent —O—CH₂—O— and each of R1, R4, R5 and R6 is hydrogen, X is not unsubstituted piperidine,
are Cavx channel blockers and are of use in the treatment of various conditions including pain.

### FIELD OF THE INVENTION

The present invention relates to ion channel modulators, and more particularly to compounds which inhibit the interaction between the pore-forming (α) subunits of Cav voltage-gated calcium channels and accessory (Cavβ subunit) proteins and their use in the treatment of a range of conditions, including pain.

### BACKGROUND TO THE INVENTION

Voltage-dependent calcium (Cav) channels conduct calcium ions across cell membranes in response to changes in the membrane voltage and thereby can regulate cellular excitability by modulating (increasing or decreasing) the electrical activity of the cell.

Cav1.x channels are involved in both skeletal (Cav1.1) and cardiac smooth muscle contraction (Cav1.2), as well as neuroendocrine release (Cav1.3 and Cav1.4). Cav2.x channels are important in neurotransmitter release (Cav2.1 and Cav2.2) and controlling neuronal excitability (Cav2.3). Cav channels which belong to the Cav1.x and Cav2.x families are defined by their threshold for activation as high threshold and are also known as L- or N-type channels respectively. L-type Cav channels are pharmacologically defined by their sensitivity to inhibition by dihydropyridines. Cav channels which belong to the Cav3.x class (Cav3.1, Cav3.2, Cav3.3) are activated at much lower membrane voltages and are defined as low threshold or T-type calcium channels.

Cav channels are composed of an al subunit, which forms the pore-region of the channel through which Ca²⁺ ions can flow. Conserved transmembrane and pore domains of the α1 subunits are less than 40% identical between the related families (Cav1.x:Cav2.x:Cav3.x) but greater than 70% identical within a familyⁱ which leads to difficulty in identifying compounds that pharmacologically discriminate between these related Cav channel subtypes.

Cav channel β subunits (Cavβ) are intracellular proteins endogenously associated with Cav channel α1 subunits, which finely tune many of their functional and electrophysiological/kinetic propertiesⁱⁱ. Ten different genes encode voltage-gated Cav channel alpha 1 subunitsⁱⁱⁱ. To date, Cavβ subunits (Cavβ1, Cavβ2, Cavβ3, Cavβ4) have been shown to interact and regulate the functional activity of Cav1.x Cav2.x and Cav3.x channels^{iv,v,vi,vii}.

The major functions of Cavβ subunits include altering the threshold for activation and the kinetics for both activation and inactivation, as well as regulating trafficking of the Cavα1 subunit to the cell membrane. The predominant effect of the combined α-β interaction is dependent upon the nature of each of the two proteins such that combining one type of Cavα subunit with any of the β(1-4) subunits will lead to differential effects on functional expression and kinetics of the channel. Thus the β subunit potentially adds a further source of modulation of the final Cav current.

Mammalian homologues of Cav channel α subunits (Cav1.x, Cav2.x and Cav3.x) consist of four homologous domains each with six transmembrane segments. These domains can form tetrameric protein complexes that span the plasma membrane of cells and allow the passage of Ca²⁺ ions. These tetrameric protein complexes of Cav channels constitute the ion channel pore-forming domain.

Functional Cav channels consisting of a tetramer of transmembrane spanning Cav2 channel subunits may be associated with and regulated by cytosolic accessory (Cavβ) proteins that are able to modulate the function of ion channel pore-forming domains (for review, see ^{viii}).

Cavβ subunits bind to Cav channel α1 subunits through an α interaction domain (AID) located between domains I and II of the pore-forming α-1 subunit. Binding of the Cavβ subunit to the AID can increase the trafficking of the Cav channel to the cell membrane and modulate the kinetics of the Cav current.

### Cav2.2 Channels and Pain

Cav2.2 calcium channels, also known as N-type channels, are located at nerve terminals, dendrites and neuroendocrine cells and are involved in neurotransmitter release^{ix}. There is substantial evidence for their involvement in pain. ω-Conotoxin-GVIA, a specific peptide blocker of Cav2.2 blocks electrically evoked responses of dorsal horn neurons and this is enhanced in nerve-injured rats^{x}. In addition, blockade of the N-type calcium channel with ω-conotoxin-GVIA, also abolishes injury-induced wind-up and post-discharge phenomena. It is suggested that nerve injury results in either increased frequency of opening of the N-type calcium channel, or an increase in the population. Blockade of these channels is expected to decrease the enhanced excitatory neurotransmitter release that occurs after nerve injury, thus inhibiting the manifestations of enhanced pain.

Neuronal Cav2.2 channels may bind to any Cavβ subunit whereas cardiac calcium currents are of the Cav1.2 type and their activity appears to be modulated by Cavβ2 proteins^{xi}. The presence of Cav2.2 with Cavβ2 produces non-inactivating currents in chromaffin cells^{xii} whereas the association of Cav2.2 with Cavβ3 produces inactivating currents. Cav2.2 would appear to be preferentially co-localised with Cavβ3 because ω-conotoxin-GVIA binding sites are immunoprecipitated by an antibody to Cavβ3 in rabbit brain^{xiii}. Mice lacking the N-type Cavβ3 subunit show reduced levels of Cav2.2 channels with altered sensitivity to inflammatory pain when compared to wild-type^{xiv}.

Furthermore, Cavβ3 subunits hyperpolarise the voltage-dependence of activation and also hyperpolarise the voltage-dependence of steady-state inactivation of Cav2.2 channels^{xv,xvi}. These channels are located at the presynaptic terminals of nociceptive neurons in dorsal horn of the spinal cord where they regulate the release of the key pro-nociceptive neurotransmitters such as glutamate and substance P. Consistent with this, selective blockers of N-type channels can be used to ameliorate chronic pain^{xvii}. One example of chronic pain is postherpetic neuralgia (PHN), traditionally defined as the persistence of pain for more than 1 month after the disappearance of the rash associated with shingles^{xviii}. Shingles is caused by the varicella-zoster virus (VZV) and can persist for years in the dorsal root ganglia of cranial or spinal nerves after resolution of the original infection. PRIALT, the synthetic analogue of ω-conotoxin-MVIIA, is effective in patients with PHN, as well as phantom-limb pain, and HIV-related neuropathic pain who are refractory to opioids^{xix}.

Compounds which are believed to inhibit chronic pain by acting at accessory proteins and reducing hyperexcitability of calcium currents have been described. Pregabalin received Food and Drug Administration (FDA) approval on Dec. 30, 2004, for the management of neuropathic pain associated with diabetic peripheral neuropathy (DPN) and PHN. Moreover, pregabalin is approved for use as adjunctive therapy for adult patients with partial onset seizures^{xx,xxi}. Pregabalin is structurally related to gabapentin (Neurontin®; Pfizer). These compounds are thought to reduce trafficking of the Cav2 channel subunit by an interaction with another accessory subunit, called α₂-δ. Pregabalin is six-times more potent than gabapentin in binding affinity to the α₂-δ voltage-gated calcium channel^{xxii}. The manufacturer states that 50 mg of pregabalin is approximately equal to 300 mg of gabapentin. Unlike classical pore-forming domain blockers pregabalin and gabapentin alter channel function without complete blockade of the calcium channel resulting in virtually no change in systemic blood pressure or coronary blood flow changes.

### Cav2.2 Channels and Lower Urinary Tract Disorders

Overactive bladder is an unmet medical need. Symptoms of overactive bladder include increased urinary frequency, urgency, nocturia (the disturbance of night-time sleep because of the need to urinate) and accidental loss of urine (urge incontinence) due to a sudden and unstoppable need to urinate. Urge incontinence is usually associated with an overactive detrusor muscle, the smooth muscle of the bladder which contracts and causes it to empty. There is no single etiology for overactive bladder. Neurogenic overactive bladder occurs as the result of neurological damage found in a variety of disorders such as stroke, Parkinson's disease, diabetes, multiple sclerosis, peripheral neuropathy, or spinal cord lesions. In these cases, the overactivity of the detrusor muscle is termed detrusor hyperreflexia.

The physiology underlying micturition is complex and the exact mechanism causing overactive bladder is unknown. Overactive bladder may result from hypersensitivity of sensory neurons of the urinary bladder, arising from inflammatory conditions, hormonal imbalances, and prostate hypertrophy. Destruction of the sensory nerve fibres, either from a crushing injury to the sacral region of the spinal cord, or from a disease that causes damage to the dorsal root fibres as they enter the spinal cord may also lead to overactive bladder. In addition, damage to the spinal cord or brain stem causing interruption of transmitted signals may lead to abnormalities in micturition. Therefore, both peripheral and central mechanisms can contribute to overactive bladder.

Carbone et al. (2003) have previously reported the effects of gabapentin on neurogenic detrusor overactivity^{xxiii}. This study demonstrated a positive effect on symptoms and significant improvement in urodynamic parameters after treatment with gabapentin and suggested that the effects of the drug should be explored further in controlled studies in both neurogenic and normeurogenic detrusor overactivity.

Further support for a pivotal role for Cav2.2 channels in the innervation of the urinary bladder comes from the finding that nitric oxide(NO), which is released by afferent nerves, acting via a cGMP signalling pathway can modulate N-type Ca²⁺ channels in DRG neurons innervating the urinary bladder^{xxiv}. Thus Cav2.2 may exert a central role in mediating control of reflex bladder activity by NO through suppressing the excitability and/or the release of transmitters from bladder afferent nerves.

Together, these studies indicate that modulation of N-type calcium channels offers a novel approach to reducing the hyperexcitability of bladder afferents in conditions of detrusor overactivity leading to overactive bladder and urinary incontinence.

Thus novel modulators of the protein-protein interaction between Cav2.2 channels and Cavβ3 accessory proteins may offer a novel mode of reducing hyperexcitability produced by over-expression of Cav2.2. Such a reduction of hyperactivity in primary afferent neurons is anticipated to lead to an alleviation of pain and of disorders of the lower urinary tract.

“Cavx” channels consist of at least 10 members which includes one of the following mammalian channels: Cav1.1, Cav1.2, Cav1.3, Cav1.4, Cav2.1, Cav2.2, Cav2.3, Cav3.1, Cav3.2 or Cav3.3 and any mammalian or non-mammalian equivalents or variants (including splice variants) thereof.

“Cavβ” proteins may include one or more of the following mammalian subunits: Cavβ1, Cavβ2, Cavβ3, Cavβ34 and any mammalian or non-mammalian equivalents or variants (including splice variants) thereof.

At a functional level, interactions between each combination of Cavx channel and Cavβ protein may confer modulation (increasing or decreasing) of a number of features of functional Cav channels including, but not limited to (i) the transport or chaperone of Cav channels to the plasma membrane of a given cell type^{xxv,xxvi,xxvii,xxviii} and/or (ii) gating properties such as channel inactivation^{xxix}.

Cavβ subunits can also exert effects on other gating properties by mechanisms which may alter the time and voltage dependency of the open (conducting state), closed (non-conducting state) and inactivated states (non-conducting state) of Cav channels.

Furthermore, the interaction between specific Cavβ subunits and different Cav channel compositions may confer differential modulation to Cav channel currents (e.g. Cav2^{xxx}). This phenomenon may account for the wide diversity of Cav channels. However, exact subunit compositions of native Cav channels and the physiologic role that particular channels play are, in most cases, still unclear.

Cavx channel inhibitors have potential utility in the treatment, prevention, inhibition, amelioration or alleviation of symptoms of a number of conditions or disease states including:

“Lower Urinary Tract Disorders”. Lower urinary tract disorders is intended to encompass both painful (any lower urinary tract disorder involving sensations or symptoms that a patient subjectively describes as producing or resulting in pain) and non-painful lower urinary tract disorders (any lower urinary tract disorder involving sensations or symptoms, including mild or general discomfort, that is subjectively described as not producing or resulting in pain). “Lower urinary tract disorders” also includes any lower urinary tract disorder characterised by overactive bladder with and/or without loss of urine, urinary frequency, urinary urgency, and nocturia. Thus, lower urinary tract disorders includes overactive bladder or overactive urinary bladder (including, overactive detrusor, detrusor instability, detrusor hyperreflexia, sensory urgency and the symptoms of detrusor overactivity), urge incontinence or urinary urge incontinence, stress incontinence or urinary stress incontinence, lower urinary tract symptoms including obstructive urinary symptoms such as slow urination, dribbling at the end of urination, inability to urinate and/or the need to strain to urinate at an acceptable rate or irritate symptoms such as frequency and/or urgency. Lower urinary tract disorders may also include neurogenic bladder that occurs as the result of neurological damage due to disorders including but not limited to stroke, Parkinson's disease, diabetes, multiple sclerosis, peripheral neuropathy, or spinal cord lesions. Lower urinary tract disorders may also include prostatitis, interstitial cystitis, benign prostatic hyperplasia, and, in spinal cord injured patients, spastic bladder.

“Anxiety and Anxiety-Related Conditions”. Anxiety and anxiety related conditions is intended to include, but is not limited to, anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias. Specific anxiety related phobias include, but are not limited to, fear of animals, insects, storms, driving, flying, heights or crossing bridges, closed or narrow spaces, water, blood or injury, as well as extreme fear of inoculations or other invasive medical or dental procedures.

“Epilepsy”. Epilepsy is intended to include, but is not limited to, one or more of the following seizures: simple partial seizures, complex partial seizures, secondary generalised seizures, generalised seizures including absence seizures, myoclonic seizures, clonic seizures, tonic seizures, tonic clonic seizures and atonic seizures.

“Pain Disorders”. Pain is intended to include but is not limited to one or more on the following: acute pain such as musculoskeletal pain, post operative pain and surgical pain; chronic pain such as chronic inflammatory pain (e.g. rheumatoid arthritis and osteoarthritis), neuropathic pain (e.g. post herpetic neuralgia, trigeminal neuralgia and sympathetically maintained pain) and pain associated with cancer and fibromyalgia; pain associated with migraine; pain (both chronic and acute), and/or fever and/or inflammation of conditions such as rheumatic fever; symptoms associated with influenza or other viral infections, such as the common cold; lower back and neck pain; headache; toothache; sprains and strains; myositis; neuralgia; synovitis; arthritis, including rheumatoid arthritis; degenerative joint diseases, including osteoarthritis; gout and ankylosing spondylitis; tendinitis; bursitis; skin-related conditions, such as psoriasis, eczema, burns and dermatitis; injuries, such as sports injuries and those arising from surgical and dental procedures.

“Cardiovascular Diseases” such as angina pectoris, hypertension and congestive heart failure.

“Gynaecological Pain”, for example, dysmenorrhoea, labour pain and pain associated with endometriosis.

“Disorders of the Auditory System” such as tinnitus.

“Migraine”.

“Gastrointestinal Disorders” including reflux esophagitis, functional dyspepsia, motility disorders (including constipation and diarrhoea), and irritable bowel syndrome.

“Vascular and Visceral Smooth Muscle Disorders” including asthma, pulmonary hypertension, chronic obstructive pulmonary disease, adult respiratory distress syndrome, peripheral vascular disease (including intermittent claudication), venous insufficiency, impotence, cerebral and coronary spasm and Raynaud's disease.

“Cell Proliferative Disorders” including restenosis and cancer (including leukaemia); treating or preventing gliomas including those of lower and higher malignancy.

“Metabolic Disorders” such as diabetes (including diabetic retinopathy, diabetic nephropathy and diabetic neuropathy), insulin resistance/insensitivity and obesity.

“Memory Loss” including Alzheimer's disease and dementia.

Other “CNS-Mediated Motor Dysfunction Disorders” including Parkinson's disease and ataxia.

“Ophthalmic Disorders” such as ocular hypertension.

It has previously been disclosed that some indole derivatives can be of use in the treatment and prevention of certain conditions, specifically certain cancers and asthma and other allergic conditions (see U.S. Pat. No. 6,693,119 and WO 98/09946). However, the scope of the compounds disclosed therein is limited, there is no suggestion that the compounds have activity as Cavx blockers and therefore there is no suggestion that they and other indoles might be of use in treating conditions where inhibition of Cavx channels will lead to treatment or prevention of said conditions.

Thus, it would be desirable to identify Cavx channel blockers for the prophylaxis or treatment of a number of disease states including lower urinary tract disorders and pain indications.

### DESCRIPTION OF THE INVENTION

In a first aspect of the present invention, there is provided a compound represented by the general formula (1) or a pharmacologically acceptable salt or pro-drug thereof, wherein:

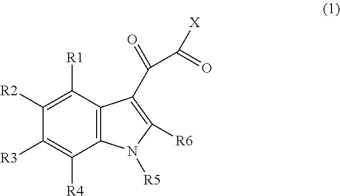

R1, R2, R3 and R4 are independently selected from hydrogen atoms, alkyl groups, hydroxyalkyl groups, halogen atoms, haloalkyl groups, alkoxy groups, haloalkoxy groups, alkoxycarbonyl groups, carboxyl groups, hydroxyl groups, nitro groups, amino groups, monalkylamino groups, dialkylamino groups, acylamino groups, alkoxycarbonylamino groups, alkylsulphonyl groups, arylsulphonyl groups, alkylsulphonylamino groups, arylsulphonylamino groups, aminosulphonyl groups and cyano groups, or

any two of R1 to R4 that are adjacent on the ring may together represent the moiety —O—(CH₂)ₙ—O— wherein n is an integer of from 1 to 3;
R5 is a hydrogen atom or an alkyl group;
R6 is a hydrogen atom or an alkyl group; and
X is selected from the group consisting of:
(a) groups of formula OR7 wherein R7 is a hydrogen atom or an alkyl group which is optionally substituted with a substituent selected from the group consisting of alkylsulfonylalkyl groups, saturated or partially unsaturated heterocyclic groups, alkoxy groups, carboxyl groups, nitro groups, amino groups, monalkylamino groups, dialkylamino groups, halogen atoms, and alkoxycarbonyl groups,
provided that when R7 is hydrogen or ethyl, then R1, R2, R3 and R4 cannot be selected from hydrogen atoms, halogen atoms and alkyl groups; and
(b) groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms, said saturated or partially unsaturated heterocyclic group optionally further being substituted by one or more substituents selected from the group consisting of alkyl groups, halogen atoms, haloalkyl groups, alkoxy groups, alkoxycarbonyl groups, carboxyl groups, nitro groups, amino groups, monalkylamino groups, dialkylamino groups and hydroxyl groups,
provided that:
(i) when R8 and R9 together with the nitrogen atom to which they are attached form a piperazine group, and one or more of R1 to R4 are selected from hydrogen atoms, hydroxyl groups, nitro groups, amino groups, alkylamino groups, dialkylamino groups, alkoxycarbonylamino groups, halogen atoms, alkoxy groups and alkyl groups, the nitrogen atom of the piperazine group at the 4-position of the ring can not be substituted by an alkyl group,
(ii) when each of R1, R2, R3, R4, R5 and R6 is a hydrogen atom, then X can not be an unsubstituted piperazinyl group or an unsubstituted morpholino group,
(iii) when each of R1, R2, R4, R5 and R6 is a hydrogen atom and R3 is selected from the group consisting of a hydrogen atom, a bromine atom and a hydroxyl group, then X can not be a methoxy group,
(iv) when each of R2 and R3 is a methoxy group or they together represent the moiety —O—CH₂—O— and each of R1, R4, R5 and R6 is a hydrogen atom, then X can not be an unsubstituted piperidine group, an unsubstituted morpholino group, an unsubstituted pyrrolidine group, an unsubstituted azepane group, an unsubstituted azocane group, or when R2 and R3 together represent the moiety —O—CH₂—O— and each of R1, R4, R5 and R6 is a hydrogen atom X cannot represent a 4-methylpiperazine group.

Preferred compounds of the invention include:

(2) compounds according to (1) and pharmacologically acceptable salts and prodrugs thereof wherein R1, R2, R3 and R4 are independently selected from hydrogen atoms, alkyl groups having from 1 to 6 carbon atoms, hydroxyl alkyl groups having from 1 to 6 carbon atoms, halogen atoms, haloalkyl groups having from 1 to 6 carbon atoms, alkoxy groups having from 1 to 6 carbon atoms, haloalkoxy groups having from 1 to 6 carbon atoms, alkoxycarbonyl groups comprising a carbonyl group which is substituted with an alkoxy group having from 1 to 6 carbon atoms, carboxyl groups, hydroxyl groups, nitro groups, amino groups, monalkylamino groups wherein the alkyl group has from 1 to 6 carbon atoms, dialkylamino groups wherein each alkyl group may be the same or different and has from 1 to 6 carbon atoms, acylamino groups comprising a carbonylamino group in which the carbonyl is substituted with a hydrogen atom or an alkyl group having from 1 to 6 carbon atoms, alkoxycarbonylamino groups comprising a carbonylamino group which is substituted with an alkoxy group having from 1 to 6 carbon atoms, alkylsulphonyl groups having from 1 to 6 carbon atoms, arylsulphonyl groups wherein the aryl moiety has from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein each alkyl group may be the same or different and has from 1 to 6 carbon atoms, nitro groups, acylamino groups comprising a carbonylamino group in which the carbonyl is substituted with a hydrogen atom or an alkyl group having from 1 to 6 carbon atoms, alkoxycarbonylamino groups comprising a carbonylamino group which is substituted with an alkoxy group having from 1 to 6 carbon atoms, alkylsulphonyl groups having from 1 to 6 carbon atoms, alkylsulphonylamino groups having from 1 to 6 carbon atoms and cyano groups, alkylsulphonylamino groups having from 1 to 6 carbon atoms, arylsulphonylamino groups wherein the aryl moiety has from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein each alkyl group may be the same or different and has from 1 to 6 carbon atoms, nitro groups, acylamino groups comprising a carbonylamino group in which the carbonyl is substituted with a hydrogen atom or an alkyl group having from 1 to 6 carbon atoms, alkoxycarbonylamino groups comprising a carbonylamino group which is substituted with an alkoxy group having from 1 to 6 carbon atoms, alkylsulphonyl groups having from 1 to 6 carbon atoms, alkylsulphonylamino groups having from 1 to 6 carbon atoms and cyano groups, aminosulphonyl groups and cyano groups, or
any two of R1 to R4 that are adjacent on the ring may together represent the moiety —O—(CH₂)ₙ—O— wherein n is 1 or 2;
(3) compounds according to (1) and pharmacologically acceptable salts and prodrugs thereof wherein R1, R2, R3 and R4 are independently selected from hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH₂—O—;
(4) compounds according to (1) and pharmacologically acceptable salts and prodrugs thereof wherein R1, R2, R3 and R4 are independently selected from hydrogen atoms, methyl groups, ethyl groups, i-propyl groups, methoxy groups, ethoxy groups, trifluoromethyl groups, fluorine atoms, chlorine atoms, bromine atoms, trifluoromethoxy groups, hydroxymethyl groups, hydroxyl groups, cyano groups and methylsulphonyl groups or any two of R1 to R4 that are adjacent on the ring may together represent the moiety —O—CH₂—O—;
(5) compounds according to any one of (1) to (4) and pharmacologically acceptable salts and prodrugs thereof wherein R5 is hydrogen or an alkyl group having from 1 to 6 carbon atoms;
(6) compounds according to any one of (1) to (4) and pharmacologically acceptable salts and prodrugs thereof wherein R5 is hydrogen or a methyl group;
(7) compounds according to any one of (1) to (6) and pharmacologically acceptable salts and prodrugs thereof wherein R6 is hydrogen or an alkyl group having from 1 to 6 carbon atoms;
(8) compounds according to any one of (1) to (6) and pharmacologically acceptable salts and prodrugs thereof wherein R6 is hydrogen or a methyl group;
(9) compounds according to any one of (1) to (8) and pharmacologically acceptable salts and prodrugs thereof wherein X is a group of formula OR7 wherein R7 is a hydrogen atom or an alkyl group having from 1 to 6 carbon atoms which may optionally be substituted with a substitutent selected from the group consisting of alkylsulfonylalkyl groups comprising alkyl groups having from 1 to 6 carbon atoms which are substituted with alkylsulfonyl groups having from 1 to 6 carbon atoms, saturated or partially unsaturated heterocyclic groups containing at least one nitrogen, oxygen or sulphur atom which are 4- to 14-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 6 carbon atoms, halogen atoms, alkoxy groups having from 1 to 6 carbon atoms, haloalkyl groups having from 1 to 6 carbon atoms and carbonyl groups, alkoxy groups having from 1 to 6 carbon atoms, carboxyl groups, nitro groups, amino groups, monalkylamino groups wherein the alkyl groups having from 1 to 6 carbon atoms, dialkylamino groups wherein each alkyl group is the same or different and each has from 1 to 6 carbon atoms, halogen atoms and alkoxycarbonyl groups comprising carbonyl groups substituted by alkoxy groups having from 1 to 6 carbon atoms,
provided that when R7 is hydrogen or ethyl, then R1, R2, R3 and R4 cannot be selected from hydrogen atoms, halogen atoms and alkyl groups;
(10) compounds according to any one of (1) to (8) and pharmacologically acceptable salts and prodrugs thereof wherein X is a group of formula OR7 wherein R7 is a hydrogen atom or an alkyl group having from 1 to 4 carbon atoms which may optionally be substituted with a substitutent selected from the group consisting of alkylsulfonylalkyl groups comprising alkyl groups having from 1 to 4 carbon atoms which are substituted with alkylsulfonyl groups having from 1 to 4 carbon atoms and saturated or partially unsaturated heterocyclic groups containing at least one nitrogen, oxygen or sulphur atom which are 4- to 8-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 4 carbon atoms, halogen atoms and alkoxy groups having from 1 to 4 carbon atoms,
provided that when R7 is hydrogen or ethyl, then R1, R2, R3 and R4 cannot be selected from hydrogen atoms, halogen atoms and alkyl groups;
(11) compounds according to any one of (1) to (8) and pharmacologically acceptable salts and prodrugs thereof wherein X is a group of formula OR7 wherein R7 is a hydrogen atom, an ethyl group, a methylsulfonylethyl group or a 2-morpholin-4-ylethyl group,
provided that when R7 is hydrogen or ethyl, then R1, R2, R3 and R4 cannot be selected from hydrogen atoms, halogen atoms and alkyl groups;
(12) compounds according to any one of (1) to (8) and pharmacologically acceptable salts and prodrugs thereof wherein X is a group of formula NR8R9 wherein R8 and R9 together with the nitrogen atom to which they are attached form a 4- to 14-membered nitrogen-containing saturated or partially unsaturated heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups,
provided that:
(i) when R8 and R9 together with the nitrogen atom to which they are attached form a piperazine group, and one or more of R1 to R4 are selected from hydrogen atoms, hydroxyl groups, nitro groups, amino groups, alkylamino groups wherein the alkyl groups have from 1 to 6 carbon atoms, dialkylamino groups wherein each alkyl group is the same or different and each is an alkyl group having from 1 to 6 carbon atoms, alkoxycarbonylamino groups comprising a carbonylamino group which is substituted with an alkoxy group having from 1 to 6 carbon atoms, halogen atoms, alkoxy groups having from 1 to 6 carbon atoms and alkyl groups having from 1 to 6 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group,
(iii) when each of R1, R2, R4, R5 and R6 is a hydrogen atom and R3 is selected from the group consisting of a hydrogen atom, a bromine atom and a hydroxyl group, then X can not be a methoxy group,
(iv) when each of R2 and R3 is a methoxy group or they together represent the moiety —O—CH₂—O— and each of R1, R4, R5 and R6 is a hydrogen atom, then X can not be an unsubstituted piperidine group, an unsubstituted morpholino group, an unsubstituted pyrrolidine group, an unsubstituted azepane group, an unsubstituted azocane group, or when R2 and R3 together represent the moiety —O—CH₂—O— and each of R1, R4, R5 and R6 is a hydrogen atom X cannot represent a 4-methylpiperazine group;
(13) compounds according to any one of (1) to (8) and pharmacologically acceptable salts and prodrugs thereof wherein X is a group of formula NR8R9 wherein R8 and R9 together with the nitrogen atom to which they are attached form a 4- to 8-membered saturated or partially unsaturated nitrogen-containing heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 4 carbon atoms, halogen atoms, haloalkyl groups having from 1 to 4 carbon atoms, alkoxy groups having from 1 to 4 carbon atoms, alkoxycarbonyl groups comprising carbonyl groups that are substituted by alkoxy groups having from 1 to 4 carbon atoms, carboxyl groups, nitro groups, amino groups, monalkylamino groups wherein the alkyl groups have from 1 to 4 carbon atoms, dialkylamino groups wherein each alkyl group is the same or different and each is an alkyl group having from 1 to 4 carbon atoms and hydroxyl groups,
(i) when R8 and R9 together with the nitrogen atom to which they are attached form a piperazine group, and one or more of R1 to R4 are selected from hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group,
(iii) when each of R1, R2, R4, R5 and R6 is a hydrogen atom and R3 is selected from the group consisting of a hydrogen atom, a bromine atom and a hydroxyl group, then X can not be a methoxy group,
(iv) when each of R2 and R3 is a methoxy group or they together represent the moiety —O—CH₂—O— and each of R1, R4, R5 and R6 is a hydrogen atom, then X can not be an unsubstituted piperidine group, an unsubstituted morpholino group, an unsubstituted pyrrolidine group, an unsubstituted azepane group, an unsubstituted azocane group, or when R2 and R3 together represent the moiety —O—CH₂—O— and each of R1, R4, R5 and R6 is a hydrogen atom X cannot represent a 4-methylpiperazine group;
(14) compounds according to any one of (1) to (8) and pharmacologically acceptable salts and prodrugs thereof wherein X is a group of formula NR8R9 wherein R8 and R9 together with the nitrogen atom to which they are attached form a 4- to 8-membered nitrogen-containing saturated heterocyclic group having one or more rings (including bridged saturated or partially unsaturated heterocyclic groups having one or more rings), which optionally further comprises an additional nitrogen, oxygen or sulphur atom, said saturated heterocyclic group optionally being substituted by one or more substituents selected from hydroxyl groups, methyl groups and ethyl groups,
(i) when R8 and R9 together with the nitrogen atom to which they are attached form a piperazine group, and one or more of R1 to R4 are selected from hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group,
(iii) when each of R1, R2, R4, R5 and R6 is a hydrogen atom and R3 is selected from the group consisting of a hydrogen atom, a bromine atom and a hydroxyl group, then X can not be a methoxy group,
(iv) when each of R2 and R3 is a methoxy group or they together represent the moiety —O—CH₂—O— and each of R1, R4, R5 and R6 is a hydrogen atom, then X can not be an unsubstituted piperidine group, an unsubstituted morpholino group, an unsubstituted pyrrolidine group, an unsubstituted azepane group, an unsubstituted azocane group, or when R2 and R3 together represent the moiety —O—CH₂—O— and each of R1, R4, R5 and R6 is a hydrogen atom X cannot represent a 4-methylpiperazine group;
(15) A compound according to any one of (1) to (8) or a pharmacologically acceptable salt or prodrug thereof wherein X is a group of formula NR8R9 wherein R8 and R9 together with the nitrogen atom to which they are attached form a morpholine ring, a 4-hydroxypiperidine ring, a piperazine ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring,
provided that when each of R1, R2, R3, R4, R5 and R6 is a hydrogen atom, then X can not be piperazine or morpholine;
(16) compounds according to (1) and pharmacologically acceptable salts and prodrugs thereof wherein:
R1, R2, R3 and R4 are independently selected from hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH₂—O—;
R5 is hydrogen or an alkyl group having from 1 to 6 carbon atoms;
R6 is hydrogen or an alkyl group having from 1 to 6 carbon atoms; and
X is a group of formula OR7 wherein R7 is a hydrogen atom or an alkyl group having from 1 to 4 carbon atoms which may optionally be substituted with a substituent selected from the group consisting of alkylsulfonylalkyl groups comprising alkyl groups having from 1 to 4 carbon atoms which are substituted with alkylsulfonyl groups having from 1 to 4 carbon atoms and saturated or partially unsaturated heterocyclic groups containing at least one nitrogen, oxygen or sulphur atom which are 4- to 8-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 4 carbon atoms, halogen atoms and alkoxy groups having from 1 to 4 carbon atoms,
provided that when R7 is hydrogen or ethyl, then R1, R2, R3 and R4 cannot be selected from hydrogen atoms, halogen atoms and alkyl groups;
(17) compounds according to (1) and pharmacologically acceptable salts and prodrugs thereof wherein:
R1, R2, R3 and R4 are independently selected from hydrogen atoms, methyl groups, ethyl groups, i-propyl groups, methoxy groups, ethoxy groups, trifluoromethyl groups, fluorine atoms, chlorine atoms, bromine atoms, trifluoromethoxy groups, hydroxymethyl groups, hydroxyl groups, cyano groups and methylsulphonyl groups or any two of R1 to R4 that are adjacent on the ring may together represent the moiety —O—CH₂—O—;
R5 is hydrogen or a methyl group;
R6 is hydrogen or a methyl group; and
X is a group of formula OR7 wherein R7 is a hydrogen atom, an ethyl group, a methylsulfonylethyl group or a 2-morpholin-4-ylethyl group,
provided that when R7 is hydrogen or ethyl, then R1, R2, R3 and R4 cannot be selected from hydrogen atoms, fluorine atoms, bromine atoms, chlorine atoms, methyl groups and ethyl groups;
(18) compounds according to (1) and pharmacologically acceptable salts and prodrugs thereof wherein:
R1, R2, R3 and R4 are independently selected from hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH₂—O—;
R5 is hydrogen or an alkyl group having from 1 to 6 carbon atoms;
R6 is hydrogen or an alkyl group having from 1 to 6 carbon atoms; and
X is a group of formula NR8R9 wherein R8 and R9 together with the nitrogen atom to which they are attached form a 4- to 8-membered nitrogen-containing saturated heterocyclic group having one or more rings (including bridged saturated or partially unsaturated heterocyclic groups having one or more rings), which optionally further comprises an additional nitrogen, oxygen or sulphur atom, said saturated heterocyclic group optionally being substituted by one or more substituents selected from hydroxyl groups, methyl groups and ethyl groups,
provided that:
(i) when R8 and R9 together with the nitrogen atom to which they are attached form a piperazine group, and one or more of R1 to R4 are selected from hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group,
(iii) when each of R1, R2, R4, R5 and R6 is a hydrogen atom and R3 is selected from the group consisting of a hydrogen atom, a bromine atom and a hydroxyl group, then X can not be a methoxy group,
(iv) when each of R2 and R3 is a methoxy group or they together represent the moiety —O—CH₂—O— and each of R1, R4, R5 and R6 is a hydrogen atom, then X can not be an unsubstituted piperidine group, an unsubstituted morpholino group, an unsubstituted pyrrolidine group, an unsubstituted azepane group, an unsubstituted azocane group, or when R2 and R3 together represent the moiety —O—CH₂—O— and each of R1, R4, R5 and R6 is a hydrogen atom X cannot represent a 4-methylpiperazine group;
(19) compounds according to (1) and pharmacologically acceptable salts and prodrugs thereof wherein:
R1, R2, R3 and R4 are independently selected from hydrogen atoms, methyl groups, ethyl groups, i-propyl groups, methoxy groups, ethoxy groups, trifluoromethyl groups, fluorine atoms, chlorine atoms, bromine atoms, trifluoromethoxy groups, hydroxymethyl groups, hydroxyl groups, cyano groups and methylsulphonyl groups or any two of R1 to R4 that are adjacent on the ring may together represent the moiety —O—CH₂—O—;
R5 is hydrogen or a methyl group;
R6 is hydrogen or a methyl group; and
X is a group of formula NR8R9 wherein R8 and R9 together with the nitrogen atom to which they are attached form a morpholine ring, a 4-hydroxypiperidine ring, a piperazine ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring,
provided that when each of R1, R2, R3, R4, R5 and R6 is a hydrogen atom, then X can not be piperazine or morpholine;
(20) A compound of formula (1) or a pharmacologically acceptable salt or prodrug thereof according to claim 1 selected from:
2-oxo-2-(6-trifluoromethoxy-1H-indol-yl)acetic acid,
2-(6-hydroxymethyl-1H-indol-3-yl)-2-oxoacetic acid,
(2-morpholin-4-ylethyl) 2-(6-methoxy-1H-indol-3-yl)-2-oxoacetate,
(2-methanesulfonylethyl)2-(6-methoxy-1H-indol-3-yl)-2-oxoacetate,
2-(7-fluoro-1H-indol-3-yl)-2-oxoacetic acid,
2-(6-methyl-1H-indol-3-yl)-2-oxoacetic acid,
2-(6-isopropyl-1H-indol-3-yl)-2-oxoacetic acid,
2-oxo-2-(6-(trifluoromethyl)-1H-indol-3-yl)acetic acid,
2-(5,6-dimethoxy-1H-indol-3-yl)-2-oxoacetic acid,
2-(6-ethoxy-1H-indol-3-yl)-2-oxoacetic acid,
2-(6-hydroxy-1H-indol-3-yl)-2-oxoacetic acid,
2-(6-cyano-1H-indol-3-yl)-2-oxoacetic acid,
2-(6-(methylsulfonyl)-1H-indol-3-yl)-2-oxoacetic acid,
ethyl 2-oxo-2-(6-(trifluoromethyl)-1H-indol-3-yl)acetate, and
ethyl 2-oxo-2-(6-(trifluoromethoxy)-1H-indol-3-yl)acetate.
   (21) A compound of formula (1) or a pharmacologically acceptable salt or prodrug thereof according to claim 1 selected from:
1-(4-hydroxypiperidin-1-yl)-2-(6-methoxy-1H-indol-3-yl)-ethane-1,2-dione,
1-(5-fluoro-1H-indol-3-yl)-2-morpholinoethane-1,2-dione,
1-(6-fluoro-1H-indol-3-yl)-2-morpholinoethane-1,2-dione,
1-(7-fluoro-1H-indol-3-yl)-2-morpholinoethane-1,2-dione,
1-(6-chloro-1H-indol-3-yl)-2-morpholinoethane-1,2-dione,
1-(5-bromo-1H-indol-3-yl)-2-morpholinoethane-1,2-dione,
1-(1-methyl-1H-indol-3-yl)-2-morpholinoethane-1,2-dione,
1-(6-methyl-1H-indol-3-yl)-2-morpholinoethane-1,2-dione,
1-(2-methyl-1H-indol-3-yl)-2-morpholinoethane-1,2-dione,
1-(6-ethyl-1H-indol-3-yl)-2-morpholinoethane-1,2-dione,
1-(6-isopropyl-1H-indol-3-yl)-2-morpholinoethane-1,2-dione,
1-morpholino-2-(6-(trifluoromethyl)-1H-indol-3-yl)ethane-1,2-dione,
1-(5-methoxy-1H-indol-3-yl)-2-morpholinoethane-1,2-dione,
1-(6-methoxy-1H-indol-3-yl)-2-morpholinoethane-1,2-dione,
1-(6-ethoxy-1H-indol-3-yl)-2-morpholinoethane-1,2-dione,
1-(5,6-dimethoxy-1H-indol-3-yl)-2-morpholinoethane-1,2-dione,
1-(5H-[1,3]dioxolo[4,5-f]indol-7-yl)-2-morpholinoethane-1,2-dione,
1-morpholine-2-(6-(trifluoromethoxy)-1H-indol-3-yl)ethane-1,2-dione,
1-(6-(methylsulfonyl)-1H-indol-3-yl)-2-morpholinoethane-1,2-dione,
1-(4-methylpiperazin-1-yl)-2-(6-(trifluoromethoxy)-1H-indol-3-yl)ethane-1,2-dione,
1-(4-methylpiperazin-1-yl)-2-(6-(trifluoromethyl)-1H-indol-3-yl)ethane-1,2-dione,
1-(6-methoxy-1H-indol-3-yl)-2-piperazin-1-yl-ethane-1,2-dione,
1-(2-methyl-1H-indol-3-yl)-2-(4-methylpiperazin-1-yl)ethane-1,2-dione,
1-(5H-[1,3]dioxolo[4,5-f]indol-7-yl)-2-(4-methylpiperazin-1-yl)ethane-1,2-dione,
1-(4-methylpiperazin-1-yl)-2-(6-(methylsulfonyl)-1H-indol-3-yl)ethane-1,2-dione,
3-[2-(4-methylpiperazin-1-yl)-2-oxo-acetyl]-1H-indole-6-carbonitrile,
1-(6-methoxy-1H-indol-3-yl)-2-(3-methyl-3,8-diaza-bicyclo[3.2.1]oct-8-yl)-ethane-1,2-dione, and
1-(6-methoxy-1H-indol-3-yl)-2-(8-methyl-3,8-diaza-bicyclo[3.2.1]oct-3-yl)-ethane-1,2-dione.
   (22) An N-Me piperazine compound or a pharmacologically acceptable salt or prodrug thereof selected from:
1-(6-hydroxy-1H-indol-3-yl)-2-(4-methylpiperazin-1-yl)ethane-1,2-dione,
1-(4-fluoro-1H-indol-3-yl)-2-(4-methylpiperazin-1-yl)ethane-1,2-dione,
1-(5-fluoro-1H-indol-3-yl)-2-(4-methylpiperazin-1-yl)ethane-1,2-dione,
1-(6-fluoro-1H-indol-3-yl)-2-(4-methylpiperazin-1-yl)ethane-1,2-dione,
1-(7-fluoro-1H-indol-3-yl)-2-(4-methylpiperazin-1-yl)ethane-1,2-dione,
1-(6-chloro-1H-indol-3-yl)-2-(4-methylpiperazin-1-yl)ethane-1,2-dione,
1-(1-methyl-1H-indol-3-yl)-2-(4-methylpiperazin-1-yl)ethane-1,2-dione,
1-(6-methyl-1H-indol-3-yl)-2-(4-methylpiperazin-1-yl)ethane-1,2-dione,
1-(6-ethyl-1H-indol-3-yl)-2-(4-methylpiperazin-1-yl)ethane-1,2-dione,
1-(6-isopropyl-1H-indol-3-yl)-2-(4-methylpiperazin-1-yl)ethane-1,2-dione,
1-(5-methoxy-1H-indol-3-yl)-2-(4-methylpiperazin-1-yl)ethane-1,2-dione,
1-(6-methoxy-1H-indol-3-yl)-2-(4-methylpiperazin-1-yl)ethane-1,2-dione
1-(6-ethoxy-1H-indol-3-yl)-2-(4-methylpiperazin-1-yl)ethane-1,2-dione, and
1-(4-methylpiperazin-1-yl)-2-(6-(methylsulfonyl)-1H-indol-3-yl)ethane-1,2-dione.

For the compounds of formula (1) of the present of the present invention, generally the most preferred compounds are those wherein X is selected from groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms, said saturated or partially unsaturated heterocyclic group optionally further being substituted by one or more substituents selected from the group consisting of alkyl groups, halogen atoms, haloalkyl groups, alkoxy groups, alkoxycarbonyl groups, carboxyl groups, nitro groups, amino groups, monalkylamino groups, dialkylamino groups and hydroxyl groups. Further particularly preferred compounds of the present invention are the N-methyl piperazine compounds of (22).

In a second aspect of the present invention, there is provided a pharmaceutical composition comprising a pharmacologically acceptable diluent or carrier and an active ingredient, wherein said active ingredient is a compound of formula (1) according to any one of (1) to (21) or an N-Me piperazine compound according to (22) or a pharmacologically acceptable salt or prodrug thereof, with the proviso that said composition does not contain 1-(1H-indol-3-yl)-2-morpholinoethane-1,2-dione.

In a third aspect of the present invention, there is provided a compound of formula (1) according to any one of (1) to (21) or an N-Me piperazine compound according to (22) or a pharmacologically acceptable salt or prodrug thereof for use as a medicament, with the proviso that said compound is not 1-(1H-indol-3-yl)-2-morpholinoethane-1,2-dione.

In a fourth aspect of the present invention, there is provided use of a compound of formula (1) according to any one of (1) to (21) or a pharmacologically acceptable salt or prodrug thereof in the preparation of a medicament for the prophylaxis or treatment of Inflammatory and Immunological Diseases.

In a fifth aspect of the present invention, there is provided use of a compound of formula (1) according to any one of (1) to (21) or a pharmacologically acceptable salt or prodrug thereof in the preparation of a medicament for the prophylaxis or treatment of Cell Proliferative Disorders.

In a sixth aspect of the present invention, there is provided use of a compound of formula (Ia) or a pharmacologically acceptable salt or prodrug thereof in the preparation of a medicament for the prophylaxis or treatment of a disease in which Cavx channels are involved, wherein:

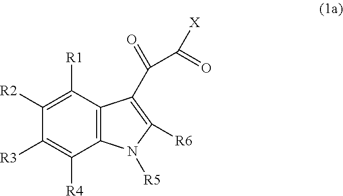

R1, R2, R3 and R4 are independently selected from hydrogen atoms, alkyl groups, hydroxyalkyl groups, halogen atoms, haloalkyl groups, alkoxy groups, haloalkoxy groups, alkoxycarbonyl groups, carboxyl groups, hydroxyl groups, nitro groups, amino groups, monalkylamino groups, dialkylamino groups, acylamino groups, alkoxycarbonylamino groups, alkylsulphonyl groups, arylsulphonyl groups, alkylsulphonylamino groups, arylsulphonylamino groups, aminosulphonyl groups and cyano groups, or
any two of R1 to R4 that are adjacent on the ring may together represent the moiety —O—(CH₂)ₙ—O— wherein n is an integer of from 1 to 3;
R5 is a hydrogen atom or an alkyl group;
R6 is a hydrogen atom or an alkyl group; and
X is selected from the group consisting of:
(a) groups of formula OR7 wherein R7 is a hydrogen atom or an alkyl group which is optionally substituted with a substituent selected from the group consisting of alkylsulfonylalkyl groups, unsaturated or partially saturated heterocyclic groups, alkoxy groups, carboxyl groups, nitro groups, amino groups, monalkylamino groups, dialkylamino groups, halogen atoms, and alkoxycarbonyl groups; and
(b) groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms, said saturated or partially unsaturated heterocyclic group optionally further being substituted by one or more substituents selected from the group consisting of alkyl groups, halogen atoms, haloalkyl groups, alkoxy groups, alkoxycarbonyl groups, carboxyl groups, nitro groups, amino groups, monalkylamino groups, dialkylamino groups and hydroxyl groups.

In a seventh aspect of the present invention, there is provided use of a compound of formula (1a) according to the sixth aspect of the present invention or a pharmacologically acceptable salt or prodrug thereof in the preparation of a medicament for the prophylaxis or treatment of a condition or disease ameliorated by Cavx channel opening.

In an eighth aspect of the present invention, there is provided use of a compound of formula (1a) according to the sixth aspect of the present invention or a pharmacologically acceptable salt or prodrug thereof in the preparation of a medicament for the prophylaxis or treatment of a condition or disease ameliorated by Cavx channel inhibition.

In a ninth aspect of the present invention, there is provided use of a compound of formula (1a) according to the sixth aspect of the present invention or a pharmacologically acceptable salt or prodrug thereof in the preparation of a medicament for the prophylaxis or treatment of Lower Urinary Tract Disorders.

In a tenth aspect of the present invention, there is provided use of a compound of formula (1a) according to the sixth aspect of the present invention or a pharmacologically acceptable salt or prodrug thereof in the preparation of a medicament for the prophylaxis or treatment of Anxiety and Anxiety-Related Conditions.

In an eleventh aspect of the present invention, there is provided use of a compound of formula (1a) according to the sixth aspect of the present invention or a pharmacologically acceptable salt or prodrug thereof in the preparation of a medicament for the prophylaxis or treatment of Epilepsy.

In a twelfth aspect of the present invention, there is provided use of a compound of formula (1a) according to the sixth aspect of the present invention or a pharmacologically acceptable salt or prodrug thereof in the preparation of a medicament for the prophylaxis or treatment of Pain Disorders.

In a thirteenth aspect of the present invention, there is provided use of a compound of formula (1a) according to the sixth aspect of the present invention or a pharmacologically acceptable salt or prodrug thereof in the preparation of a medicament for the prophylaxis or treatment of Gynaecological Pain.

In a fourteenth aspect of the present invention, there is provided use of a compound of formula (1a) according to the sixth aspect of the present invention or a pharmacologically acceptable salt or prodrug thereof in the preparation of a medicament for the prophylaxis or treatment of Cardiac Arrhythmias.

In a fifteenth aspect of the present invention, there is provided use of a compound of formula (1a) according to the sixth aspect of the present invention or a pharmacologically acceptable salt or prodrug thereof in the preparation of a medicament for the prophylaxis or treatment of Thromboembolic Events.

In a sixteenth aspect of the present invention, there is provided use of a compound of formula (1a) according to the sixth aspect of the present invention or a pharmacologically acceptable salt or prodrug thereof in the preparation of a medicament for the prophylaxis or treatment of Cardiovascular Diseases.

In a seventeenth aspect of the present invention, there is provided use of a compound of formula (1a) according to the sixth aspect of the present invention or a pharmacologically acceptable salt or prodrug thereof in the preparation of a medicament for the prophylaxis or treatment of Disorders of the Auditory System.

In an eighteenth aspect of the present invention, there is provided use of a compound of formula (1a) according to the sixth aspect of the present invention or a pharmacologically acceptable salt or prodrug thereof in the preparation of a medicament for the prophylaxis or treatment of Migraine.

In an nineteenth aspect of the present invention, there is provided use of a compound of formula (1a) according to the sixth aspect of the present invention or a pharmacologically acceptable salt or prodrug thereof in the preparation of a medicament for the prophylaxis or treatment of Gastrointestinal Disorders.

In a twentieth aspect of the present invention, there is provided use of a compound of formula (1a) according to the sixth aspect of the present invention or a pharmacologically acceptable salt or prodrug thereof in the preparation of a medicament for the prophylaxis or treatment of Vascular and Visceral Smooth Muscle Disorders.

In a twenty-first aspect of the present invention, there is provided use of a compound of formula (1a) according to the sixth aspect of the present invention or a pharmacologically acceptable salt or prodrug thereof in the preparation of a medicament for the prophylaxis or treatment of Metabolic Disorders.

In a twenty-second aspect of the present invention, there is provided use of a compound of formula (1a) according to the sixth aspect of the present invention or a pharmacologically acceptable salt or prodrug thereof in the preparation of a medicament for the prophylaxis or treatment of Memory Loss.

In a twenty-third aspect of the present invention, there is provided use of a compound of formula (1a) according to the sixth aspect of the present invention or a pharmacologically acceptable salt or prodrug thereof in the preparation of a medicament for the prophylaxis or treatment of CNS-Mediated Motor Dysfunction Disorders.

In a twenty-fourth aspect of the present invention, there is provided use of a compound of formula (1a) according to the sixth aspect of the present invention or a pharmacologically acceptable salt or prodrug thereof in the preparation of a medicament for the prophylaxis or treatment of Ophthalmic Disorders.

Preferably, in any one of the sixth to twenty-fourth aspects of the invention, X is a group of formula OR7 wherein R7 is a hydrogen atom or an alkyl group having from 1 to 6 carbon atoms which may optionally be substituted with a substituent selected from the group consisting of alkylsulfonylalkyl groups comprising alkyl groups having from 1 to 6 carbon atoms which are substituted with alkylsulfonyl groups having from 1 to 6 carbon atoms, saturated or partially unsaturated heterocyclic groups containing at least one nitrogen, oxygen or sulphur atom which are 4- to 14-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 6 carbon atoms, halogen atoms, alkoxy groups having from 1 to 6 carbon atoms, haloalkyl groups having from 1 to 6 carbon atoms and carbonyl groups, alkoxy groups having from 1 to 6 carbon atoms, carboxyl groups, nitro groups, amino groups, monalkylamino groups wherein the alkyl groups having from 1 to 6 carbon atoms, dialkylamino groups wherein each alkyl group is the same or different and each has from 1 to 6 carbon atoms, halogen atoms and alkoxycarbonyl groups comprising carbonyl groups substituted by alkoxy groups having from 1 to 6 carbon atoms, or

X is a group of formula NR8R9 wherein R8 and R9 together with the nitrogen atom to which they are attached form a 4- to 14-membered nitrogen-containing saturated or partially unsaturated heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups.

More preferably, in any one of the sixth to twenty-fourth aspects of the invention, X is a group of formula OR7 wherein R7 is a hydrogen atom or an alkyl group having from 1 to 4 carbon atoms which may optionally be substituted with a substituent selected from the group consisting of alkylsulfonylalkyl groups comprising alkyl groups having from 1 to 4 carbon atoms which are substituted with alkylsulfonyl groups having from 1 to 4 carbon atoms and saturated or partially unsaturated heterocyclic groups containing at least one nitrogen, oxygen or sulphur atom which are 4- to 8-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 4 carbon atoms, halogen atoms and alkoxy groups having from 1 to 4 carbon atoms, or

X is a group of formula NR8R9 wherein R8 and R9 together with the nitrogen atom to which they are attached form a 4- to 8-membered saturated or partially unsaturated nitrogen-containing heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 4 carbon atoms, halogen atoms, haloalkyl groups having from 1 to 4 carbon atoms, alkoxy groups having from 1 to 4 carbon atoms, alkoxycarbonyl groups comprising carbonyl groups that are substituted by alkoxy groups having from 1 to 4 carbon atoms, carboxyl groups, nitro groups, amino groups, monalkylamino groups wherein the alkyl groups have from 1 to 4 carbon atoms, dialkylamino groups wherein each alkyl group is the same or different and each is an alkyl group having from 1 to 4 carbon atoms and hydroxyl groups.

Yet more preferably, in any one of the sixth to twenty-fourth aspects of the invention, X is a group of formula OR7 wherein R7 is a hydrogen atom, an ethyl group, a methylsulfonylethyl group or a 2-morpholin-4-ylethyl group, or

X is a group of formula NR8R9 wherein R8 and R9 together with the nitrogen atom to which they are attached form a 4- to 8-membered nitrogen-containing saturated heterocyclic group having one or more rings (including bridged saturated or partially unsaturated heterocyclic groups having one or more rings), which optionally further comprises an additional nitrogen, oxygen or sulphur atom, said saturated heterocyclic group optionally being substituted by one or more substituents selected from hydroxyl groups, methyl groups and ethyl groups.

Most preferably, in any one of the sixth to twenty-fourth aspects of the invention, X is a group of formula OR7 wherein R7 is a hydrogen atom, an ethyl group, a methylsulfonylethyl group or a 2-morpholin-4-ylethyl group, or

X is a group of formula NR8R9 wherein R8 and R9 together with the nitrogen atom to which they are attached form a morpholine ring, a 4-hydroxypiperidine ring, a piperazine ring, a 4-methylpiperazin-1-yl ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring.

Particularly preferred compounds for use in any one of the sixth to twenty-fourth aspects of the invention are generally those wherein X is a group of formula NR8R9 wherein R8 and R9 together with the nitrogen atom to which they are attached form a 4- to 14-membered nitrogen-containing saturated or partially unsaturated heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups.

In the fourth and fifth aspects of the inventions, most preferably there is provided use of a compound of formula (1) according to (20) or (21) or a pharmacologically acceptable salt or prodrug thereof.

In any one of the sixth to twenty-fourth aspects of the invention, most preferably there is provided use of a compound of formula (1) according to (20) or (21), an N-Me piperazine compound according to (22), a compound selected from the list below, or a pharmacologically acceptable salt or prodrug thereof:
2-(4-fluoro-1H-indol-3-yl)-2-oxoacetic acid,
2-(5-fluoro-1H-indol-3-yl)-2-oxoacetic acid,
2-(6-fluoro-1H-indol-3-yl)-2-oxoacetic acid,
2-(6-chloro-1H-indol-3-yl)-2-oxoacetic acid,
2-(5-bromo-1H-indol-3-yl)-2-oxoacetic acid,
2-(6-bromo-1H-indol-3-yl)-2-oxoacetic acid,
2-(1-methyl-1H-indol-3-yl)-2-oxoacetic acid,
2-(2-methyl-1H-indol-3-yl)-2-oxoacetic acid,
2-(5-methoxy-1H-indol-3-yl)-2-oxoacetic acid,
2-(6-methoxy-1H-indol-3-yl)-2-oxoacetic acid,
ethyl 2-(1H-indol-3-yl)-2-oxoacetate,
ethyl 2-(6-fluoro-1H-indol-3-yl)-2-oxoacetate,
ethyl 2-(6-methoxy-1H-indol-3-yl)-2-oxoacetate,
1-(5-bromo-1H-indol-3-yl)-2-(4-methylpiperazin-1-yl)ethane-1,2-dione,
1-(2-methyl-1H-indol-3-yl)-2-(4-methylpiperazin-1-yl)ethane-1,2-dione, and
1-(5,6-dimethoxy-1H-indol-3-yl)-2-(4-methylpiperazin-1-yl)ethane-1,2-dione.

In a twenty-fifth aspect of the present invention, there is provided a method for the prophylaxis or treatment of an Inflammatory or Immunological Disease in a patient in need thereof comprising administering to said patient an effective amount of a compound of formula (1) according to any one of (1) to (21) or a pharmacologically acceptable salt or prodrug thereof.

In a twenty-sixth aspect of the present invention, there is provided a method for the prophylaxis or treatment of Cell Proliferative Disorders comprising administering to said patient an effective amount of a compound of formula (1) according to any one of (1) to (21) or a pharmacologically acceptable salt or prodrug thereof.

In a twenty-seventh aspect of the present invention, there is provided a method for the prophylaxis or treatment of a disease in which Cavx channels are involved comprising administering to said patient an effective amount of a compound of formula (Ia) or a pharmacologically acceptable salt or prodrug thereof, wherein:

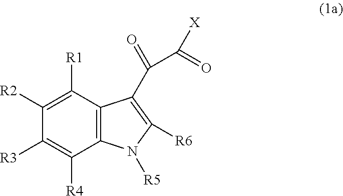

R1, R2, R3 and R4 are independently selected from hydrogen atoms, alkyl groups, hydroxyalkyl groups, halogen atoms, haloalkyl groups, alkoxy groups, haloalkoxy groups, alkoxycarbonyl groups, carboxyl groups, hydroxyl groups, nitro groups, amino groups, monalkylamino groups, dialkylamino groups, acylamino groups, alkoxycarbonylamino groups, alkylsulphonyl groups, arylsulphonyl groups, alkylsulphonylamino groups, arylsulphonylamino groups, aminosulphonyl groups and cyano groups, or
any two of R1 to R4 that are adjacent on the ring may together represent the moiety —O—(CH₂)ₙ—O— wherein n is an integer of from 1 to 3;
R5 is a hydrogen atom or an alkyl group;
R6 is a hydrogen atom or an alkyl group; and
X is selected from the group consisting of:
(a) groups of formula OR7 wherein R7 is a hydrogen atom or an alkyl group which is optionally substituted with a substituent selected from the group consisting of alkylsulfonylalkyl groups, unsaturated or partially saturated heterocyclic groups, alkoxy groups, carboxyl groups, nitro groups, amino groups, monalkylamino groups, dialkylamino groups, halogen atoms, and alkoxycarbonyl groups; and
(b) groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms, said saturated or partially saturated unsaturated heterocyclic group optionally further being substituted by one or more substituents selected from the group consisting of alkyl groups, halogen atoms, haloalkyl groups, alkoxy groups, alkoxycarbonyl groups, carboxyl groups, nitro groups, amino groups, monalkylamino groups, dialkylamino groups and hydroxyl groups.

In a twenty-eighth aspect of the present invention, there is provided a method for the prophylaxis or treatment of a condition or disease ameliorated by Cavx channel opening comprising administering to said patient an effective amount of a compound of formula (1a) according to the twenty-seventh aspect of the present invention or a pharmacologically acceptable salt or prodrug thereof.

In a twenty-ninth aspect of the present invention, there is provided a method for the prophylaxis or treatment of a condition or disease ameliorated by Cavx channel inhibition comprising administering to said patient an effective amount of a compound of formula (1a) according to the twenty-seventh aspect of the present invention or a pharmacologically acceptable salt or prodrug thereof.

In a thirtieth aspect of the present invention, there is provided a method for the prophylaxis or treatment of Lower Urinary Tract Disorders comprising administering to said patient an effective amount of a compound of formula (1a) according to the twenty-seventh aspect of the present invention or a pharmacologically acceptable salt or prodrug thereof.

In a thirty-first aspect of the present invention, there is provided a method for the prophylaxis or treatment of Anxiety and Anxiety-Related Conditions Disorders comprising administering to said patient an effective amount of a compound of formula (1a) according to the twenty-seventh aspect of the present invention or a pharmacologically acceptable salt or prodrug thereof.

In a thirty-second aspect of the present invention, there is provided a method for the prophylaxis or treatment of Epilepsy comprising administering to said patient an effective amount of a compound of formula (1a) according to the twenty-seventh aspect of the present invention or a pharmacologically acceptable salt or prodrug thereof.

In a thirty-third aspect of the present invention, there is provided a method for the prophylaxis or treatment of Pain Disorders comprising administering to said patient an effective amount of a compound of formula (1a) according to the twenty-seventh aspect of the present invention or a pharmacologically acceptable salt or prodrug thereof.

In a thirty-fourth aspect of the present invention, there is provided a method for the prophylaxis or treatment of Gynaecological Pain comprising administering to said patient an effective amount of a compound of formula (1a) according to the twenty-seventh aspect of the present invention or a pharmacologically acceptable salt or prodrug thereof.

In a thirty-fifth aspect of the present invention, there is provided a method for the prophylaxis or treatment of Cardiac Arrhythmias comprising administering to said patient an effective amount of a compound of formula (1a) according to the twenty-seventh aspect of the present invention or a pharmacologically acceptable salt or prodrug thereof.

In a thirty-sixth aspect of the present invention, there is provided a method for the prophylaxis or treatment of Thromboembolic Events comprising administering to said patient an effective amount of a compound of formula (1a) according to the twenty-seventh aspect of the present invention or a pharmacologically acceptable salt or prodrug thereof.

In a thirty-seventh aspect of the present invention, there is provided a method for the prophylaxis or treatment of Cardiovascular Diseases comprising administering to said patient an effective amount of a compound of formula (1a) according to the twenty-seventh aspect of the present invention or a pharmacologically acceptable salt or prodrug thereof.

In a thirty-eighth aspect of the present invention, there is provided a method for the prophylaxis or treatment of Disorders of the Auditory System comprising administering to said patient an effective amount of a compound of formula (1a) according to the twenty-seventh aspect of the present invention or a pharmacologically acceptable salt or prodrug thereof.

In a thirty-ninth aspect of the present invention, there is provided a method for the prophylaxis or treatment of Migraine comprising administering to said patient an effective amount of a compound of formula (1a) according to the twenty-seventh aspect of the present invention or a pharmacologically acceptable salt or prodrug thereof.

In an fortieth aspect of the present invention, there is provided a method for the prophylaxis or treatment of Gastrointestinal Disorders comprising administering to said patient an effective amount of a compound of formula (1a) according to the twenty-seventh aspect of the present invention or a pharmacologically acceptable salt or prodrug thereof.

In a forty-first aspect of the present invention, there is provided a method for the prophylaxis or treatment of Vascular and Visceral Smooth Muscle Disorders comprising administering to said patient an effective amount of a compound of formula (1a) according to the twenty-seventh aspect of the present invention or a pharmacologically acceptable salt or prodrug thereof.

In forty-second aspect of the present invention, there is provided a method for the prophylaxis or treatment of Metabolic Disorders comprising administering to said patient an effective amount of a compound formula (1a) according to the twenty-seventh aspect of the present invention or a pharmacologically acceptable salt or prodrug thereof.

In a forty-third aspect of the present invention, there is provided a method for the prophylaxis or treatment of Memory Loss comprising administering to said patient an effective amount of a compound of formula (1a) according to the twenty-seventh aspect of the present invention or a pharmacologically acceptable salt or prodrug thereof.

In a forty-fourth aspect of the present invention, there is provided a method for the prophylaxis or treatment of CNS-Mediated Motor Dysfunction Disorders comprising administering to said patient an effective amount of a compound of formula (1a) according to the twenty-seventh aspect of the present invention or a pharmacologically acceptable salt or prodrug thereof.

In a forty-fifth aspect of the present invention, there is provided a method for the prophylaxis or treatment of Ophthalmic Disorders comprising administering to said patient an effective amount of compound of formula (1a) according to the twenty-seventh aspect of the present invention or a pharmacologically acceptable salt or prodrug thereof.

Preferably, in any one of the twenty-seventh to forty-fifth aspects of the invention, X is a group of formula OR7 wherein R7 is a hydrogen atom or an alkyl group having from 1 to 6 carbon atoms which may optionally be substituted with a substituent selected from the group consisting of alkylsulfonylalkyl groups comprising alkyl groups having from 1 to 6 carbon atoms which are substituted with alkylsulfonyl groups having from 1 to 6 carbon atoms, saturated or partially unsaturated heterocyclic groups containing at least one nitrogen, oxygen or sulphur atom which are 4- to 14-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 6 carbon atoms, halogen atoms, alkoxy groups having from 1 to 6 carbon atoms, haloalkyl groups having from 1 to 6 carbon atoms and carbonyl groups, alkoxy groups having from 1 to 6 carbon atoms, carboxyl groups, nitro groups, amino groups, monalkylamino groups wherein the alkyl groups having from 1 to 6 carbon atoms, dialkylamino groups wherein each alkyl group is the same or different and each has from 1 to 6 carbon atoms, halogen atoms and alkoxycarbonyl groups comprising carbonyl groups substituted by alkoxy groups having from 1 to 6 carbon atoms, or

X is a group of formula NR8R9 wherein R8 and R9 together with the nitrogen atom to which they are attached form a 4- to 14-membered nitrogen-containing saturated or partially unsaturated heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups.

More preferably, in any one of the twenty-seventh to forty-fifth aspects of the invention, X is a group of formula OR7 wherein R7 is a hydrogen atom or an alkyl group having from 1 to 4 carbon atoms which may optionally be substituted with a substituent selected from the group consisting of alkylsulfonylalkyl groups comprising alkyl groups having from 1 to 4 carbon atoms which are substituted with alkylsulfonyl groups having from 1 to 4 carbon atoms and saturated or partially unsaturated heterocyclic groups containing at least one nitrogen, oxygen or sulphur atom which are 4- to 8-membered saturated or partially unsaturated heterocyclic groups having one or more rings (including bridged saturated or partially unsaturated heterocyclic groups having one or more rings), said saturated or partially unsaturated heterocyclic groups optionally being substituted with at least one substituent selected from alkyl groups having from 1 to 4 carbon atoms, halogen atoms and alkoxy groups having from 1 to 4 carbon atoms, or

X is a group of formula NR8R9 wherein R8 and R9 together with the nitrogen atom to which they are attached form a 4- to 8-membered saturated or partially unsaturated nitrogen-containing heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 4 carbon atoms, halogen atoms, haloalkyl groups having from 1 to 4 carbon atoms, alkoxy groups having from 1 to 4 carbon atoms, alkoxycarbonyl groups comprising carbonyl groups that are substituted by alkoxy groups having from 1 to 4 carbon atoms, carboxyl groups, nitro groups, amino groups, monalkylamino groups wherein the alkyl groups have from 1 to 4 carbon atoms, dialkylamino groups wherein each alkyl group is the same or different and each is an alkyl group having from 1 to 4 carbon atoms and hydroxyl groups.

Yet more preferably, in any one of the twenty-seventh to forty-fifth aspects of the invention, X is a group of formula OR7 wherein R7 is a hydrogen atom, an ethyl group, a methylsulfonylethyl group or a 2-morpholin-4-ylethyl group, or

X is a group of formula NR8R9 wherein R8 and R9 together with the nitrogen atom to which they are attached form a 4- to 8-membered nitrogen-containing saturated heterocyclic group having one or more rings (including bridged saturated or partially unsaturated heterocyclic groups having one or more rings), which optionally further comprises an additional nitrogen, oxygen or sulphur atom, said saturated heterocyclic group optionally being substituted by one or more substituents selected from hydroxyl groups, methyl groups and ethyl groups.

Most preferably, in any one of the twenty-seventh to forty-fifth aspects of the invention, X is a group of formula OR7 wherein R7 is a hydrogen atom, an ethyl group, a methylsulfonylethyl group or a 2-morpholin-4-ylethyl group, or

X is a group of formula NR8R9 wherein R8 and R9 together with the nitrogen atom to which they are attached form a morpholine ring, a 4-hydroxypiperidine ring, a piperazine ring, a 4-methylpiperazin-1-yl ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring.

In the twenty-fifth and twenty-sixth aspects of the inventions, most preferably said methods comprise administering to the patient in need thereof an effective amount of a compound of formula (1) according to (20) or (21) or a pharmacologically acceptable salt or prodrug thereof.

In any one of the twenty-seventh to forty-fifth aspects of the invention, most preferably said methods comprise administering to the patient in need thereof an effective amount of a compound of formula (1) according to (20) or (21), an N-Me piperazine compound according to (22), a compound selected from the list below or a pharmacologically acceptable salt or prodrug thereof:
2-(4-fluoro-1H-indol-3-yl)-2-oxoacetic acid,
2-(5-fluoro-1H-indol-3-yl)-2-oxoacetic acid,
2-(6-fluoro-1H-indol-3-yl)-2-oxoacetic acid,
2-(6-chloro-1H-indol-3-yl)-2-oxoacetic acid,
2-(5-bromo-1H-indol-3-yl)-2-oxoacetic acid,
2-(6-bromo-1H-indol-3-yl)-2-oxoacetic acid,
2-(1-methyl-1H-indol-3-yl)-2-oxoacetic acid,
2-(2-methyl-1H-indol-3-yl)-2-oxoacetic acid,
2-(5-methoxy-1H-indol-3-yl)-2-oxoacetic acid,
2-(6-methoxy-1H-indol-3-yl)-2-oxoacetic acid,
ethyl 2-(1H-indol-3-yl)-2-oxoacetate,
ethyl 2-(6-fluoro-1H-indol-3-yl)-2-oxoacetate,
ethyl 2-(6-methoxy-1H-indol-3-yl)-2-oxoacetate,
1-(5-bromo-1H-indol-3-yl)-2-(4-methylpiperazin-1-yl)ethane-1,2-dione,
1-(2-methyl-1H-indol-3-yl)-2-(4-methylpiperazin-1-yl)ethane-1,2-dione, and
1-(5,6-dimethoxy-1H-indol-3-yl)-2-(4-methylpiperazin-1-yl)ethane-1,2-dione.

In a forty-sixth aspect of the present invention, there is provided a compound of formula (1) according to any one of (1) to (21) or a pharmacologically acceptable salt or prodrug thereof for use in the prophylaxis or treatment of the diseases or conditions recited in the fourth and fifth aspects of the invention recited above.

In a forty-seventh aspect of the present invention, there is provided a compound of formula (1a) as defined in the sixth aspect of the present invention or a pharmacologically acceptable salt or prodrug thereof for use in the prophylaxis or treatment of any disease or condition recited in the sixth to twenty-fourth aspects of the invention recited above.

In a forty-eighth aspect of the present invention there is provided a pharmaceutical composition comprising a pharmacologically acceptable diluent or carrier and at least two active ingredients, wherein said active ingredients comprise at least one compound of formula (1) according to any one of (1) to (21) or a pharmacologically acceptable salt or prodrug thereof, at least one N-methyl piperazine derivative as defined in (22) or a pharmacologically acceptable salt or prodrug thereof, or a compound of formula (1a) as defined in the sixth aspect of the present invention or a pharmacologically acceptable salt or prodrug thereof in combination with at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists, alpha-1 adrenoceptor antagonists, tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

Preferred pharmaceutical combinations according to the present invention include:

(1) a pharmaceutical composition comprising a pharmacologically acceptable diluent or carrier and a combination of active ingredients, wherein said active ingredients comprise at least one compound of formula (1) according to any one of (1) to (21) or a pharmacologically acceptable salt or prodrug thereof, at least one N-methyl piperazine derivative as defined in (22) or a pharmacologically acceptable salt or prodrug thereof, or at least one compound of formula (1a) as defined in the sixth aspect of the present invention or a pharmacologically acceptable salt or prodrug thereof in combination at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists and α-1 adrenoceptor antagonists; and
(2) a pharmaceutical composition comprising a pharmacologically acceptable diluent or carrier and a combination of active ingredients, wherein said active ingredients comprise at least one compound of formula (1) according to any one of (1) to (21) or a pharmacologically acceptable salt or prodrug thereof, at least one N-methyl piperazine derivative as defined in (22) or a pharmacologically acceptable salt or prodrug thereof, or at least one compound of formula (1a) as defined in the sixth aspect of the present invention or a pharmacologically acceptable salt or prodrug thereof in combination at least one compound selected from the group consisting of neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

The combinations of preferred option (1) are of particular use in the prophylaxis or treatment of lower urinary tract disorders. The combinations of preferred option (2) are of particular use in the prophylaxis or treatment of pain.

In a forty-ninth aspect of the present invention there is provided use of at least one compound of formula (1) according to any one of (1) to (21) or a pharmacologically acceptable salt or prodrug thereof, at least one N-methyl piperazine derivative as defined in (22) or a pharmacologically acceptable salt or prodrug thereof, or at least one compound of formula (1a) as defined in the sixth aspect of the present invention or a pharmacologically acceptable salt or prodrug thereof and at least one compound selected from the group consisting of muscarinic receptor antagonists, β3 adrenergic receptor agonists, neurokinin K receptor antagonists, vanilloid VR1 agonists, calcium channel α2 δ delta ligands, potassium channel inhibitors, calcium channel inhibitors, sodium channel blockers, serotonin and norepinephrine reuptake inhibitors (SNRIs), 5-HT antagonists and α-1 adrenoceptor antagonists in the manufacture of a medicament for the prophylaxis or treatment of lower urinary tract disorders.

In a fiftieth of the present invention there is provided use of at least one compound of formula (1) according to any one of (1) to (21) or a pharmacologically acceptable salt or prodrug thereof, at least one N-methyl piperazine derivative as defined in (22) or a pharmacologically acceptable salt or prodrug thereof, or at least one compound of formula (1a) as defined in the sixth aspect of the present invention or a pharmacologically acceptable salt or prodrug thereof and at least one compound selected from the group consisting of neurokinin K receptor antagonists, vanilloid VR1 agonists, calcium channel α2 δ delta ligands, potassium channel inhibitors, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs) in the manufacture of a medicament for the prophylaxis or treatment of pain.

### DETAILED DESCRIPTION OF THE INVENTION

In the compounds of the present invention, the alkyl group in the definitions of R1, R2, R3, R4, R5, R6, R7, R8 and R9 is preferably an alkyl group having from 1 to 6 carbon atoms, more preferably an alkyl group having from 1 to 4 carbon atoms and most preferably a methyl group, an ethyl group or an i-propyl group.

In the compounds of the present invention, the arylsulphonyl group in the definitions of R1, R2, R3 and R4 is preferably an arylsulphonyl group wherein the aryl moiety has from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein each alkyl group may be the same or different and has from 1 to 6 carbon atoms, nitro groups, acylamino groups comprising a carbonylamino group in which the carbonyl is substituted with a hydrogen atom or an alkyl group having from 1 to 6 carbon atoms, alkoxycarbonylamino groups comprising a carbonylamino group which is substituted with an alkoxy group having from 1 to 6 carbon atoms, alkylsulphonyl groups having from 1 to 6 carbon atoms, alkylsulphonylamino groups having from 1 to 6 carbon atoms and cyano groups, alkylsulphonylamino groups having from 1 to 6 carbon atoms, arylsulphonylamino groups wherein the aryl moiety has from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein each alkyl group may be the same or different and has from 1 to 6 carbon atoms, nitro groups, acylamino groups comprising a carbonylamino group in which the carbonyl is substituted with a hydrogen atom or an alkyl group having from 1 to 6 carbon atoms. Examples of the unsubstituted arylsulphonyl groups include phenylsulphonyl, indenylsulphonyl, naphthylsulphonyl, phenanthrenylsulphonyl and anthracenylsulphonyl groups. More preferred arylsulphonyl groups include phenylsulphonyl groups which may optionally substituted by 1 or 2 alkyl groups having from 1 to 6 carbon atoms.

In the compounds of the present invention, the haloalkyl group in the definitions of R1, R2, R3, R4, R8 and R9 is preferably an alkyl group as defined above which is substituted with one or more halogen atoms. More preferably, it is an alkyl group having from 1 to 4 carbon atoms that is substituted with at least one chlorine or fluorine atom and most preferably it is a chloromethyl group, a trichloromethyl group, a trifluoromethyl group or a tetrafluoroethyl group.

In the compounds of the present invention, the alkoxy group in the definitions of R1, R2, R3, R4, R7, R8 and R9 is preferably an alkoxy group having from 1 to 6 carbon atoms, more preferably an alkoxy group having from 1 to 4 carbon atoms and most preferably a methoxy or ethoxy group.

In the compounds of the present invention, the haloalkoxy group in the definitions of R1, R2, R3 and R4, is preferably an alkoxy group as defined above that is substituted by one or more halogen atoms, more preferably a haloalkoxy group having from 1 to 4 carbon atoms substituted by one or more chlorine or fluorine atoms and most preferably a chloromethoxy group, a trichloromethoxy group, a trifluoromethoxy group or a pentafluoroethoxy group.

In the compounds of the present invention, the hydroxyalkyl group in the definition of R1, R2, R3 and R4 is preferably an alkyl group as defined above which is substituted by at least one hydroxy group, more preferably an alkyl group having from 1 to 4 carbon atoms which is substituted with a hydroxy group and most preferably a hydroxylmethyl group or a 2-hydroxyethyl group.

In the compounds of the present invention, the alkoxycarbonyl group in the definitions of R1, R2, R3, R4, R7, R8 and R9 is preferably a carbonyl group which is substituted with an alkoxy group as defined above, and is more preferably a methoxycarbonyl or ethoxycarbonyl group.

In the compounds of the present invention, the alkoxycarbonylamino group in the definitions of R1, R2, R3 and R4, is preferably an amino group which is substituted with an alkoxycarbonyl group as defined above, and is more preferably a methoxycarbonylamino or ethoxycarbonylamino group.

In the compounds of the present invention, the monalkylamino group in the definitions of R1, R2, R3, R4, R7, R8 and R9 is preferably an amino group which is substituted with one alkyl group as defined above, and is more preferably a methylamino, ethylamino or t-butylamino group.

In the compounds of the present invention, the dialkylamino group in the definitions of R1, R2, R3, R4, R7, R8 and R9 is preferably an amino group which is substituted with two alkyl groups as defined above which may be the same or different from each other, and is more preferably a dimethylamino or diethylamino group.

In the compounds of the present invention, the acylamino group in the definitions of R1, R2, R3 and R4 is preferably an amino group which is substituted with an acyl group having from 1 to 6 carbon atoms and is more preferably an acetylamino or propanoylamino group.

In the compounds of the present invention, the alkylsulphonyl group in the definitions of R1, R2, R3 and R4 is preferably a sulphonyl group which is substituted with an alkyl group as defined above and is more preferably a methylsulphonyl or ethylsulphonyl group.

In the compounds of the present invention, the alkylsulphonylamino group in the definitions of R1, R2, R3 and R4 is preferably an amino group which is substituted with an alkylsulphonyl group as defined above and is more preferably a methylsulphonylamino or ethylsulphonylamino group.

In the compounds of the present invention, the saturated or partially unsaturated heterocyclic group in the definition of R7 is preferably a 4- to 14-membered saturated or partially unsaturated heterocyclic group having one or more rings (including bridged saturated or partially unsaturated heterocyclic groups having one or more rings), said saturated or partially unsaturated heterocyclic group optionally being substituted with at least one substituent selected from alkyl groups having from 1 to 6 carbon atoms, halogen atoms, alkoxy groups having from 1 to 6 carbon atoms, haloalkyl groups having from 1 to 6 carbon atoms and carbonyl groups, alkoxy groups having from 1 to 6 carbon atoms, carboxyl groups, nitro groups, amino groups, monalkylamino groups wherein the alkyl groups having from 1 to 6 carbon atoms, dialkylamino groups wherein each alkyl group is the same or different and each has from 1 to 6 carbon atoms, halogen atoms and alkoxycarbonyl groups comprising carbonyl groups substituted by alkoxy groups having from 1 to 6 carbon atoms, more preferably a 4- to 8-membered saturated or partially unsaturated heterocyclic group containing at least one nitrogen, oxygen or sulphur atom having one or more rings, including bridged-ring groups, said saturated or partially unsaturated heterocyclic group optionally being substituted with at least one substituent selected from alkyl groups having from 1 to 4 carbon atoms, halogen atoms and alkoxy groups having from 1 to 4 carbon atoms, and most preferably a morpholin-4-yl group.

In the compounds of the present invention, the saturated or partially unsaturated heterocyclic group in the definition of NR8R9 is preferably a 4- to 14-membered nitrogen-containing saturated or partially unsaturated heterocyclic group having one or more rings (including bridged saturated or partially unsaturated heterocyclic groups having one or more rings), which optionally further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups, more preferably the saturated or partially unsaturated heterocyclic group in the definition of NR8R9 is a 4- to 8-membered nitrogen-containing saturated heterocyclic group having one or more rings, including bridged-ring saturated heterocyclic groups, which may further comprise an additional nitrogen, oxygen or sulphur atom, said saturated heterocyclic group optionally being substituted by one or more substituents selected from hydroxyl groups, methyl groups and ethyl groups, and most preferably it is a morpholine ring, a 4-hydroxypiperidine ring, a piperazine ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring and, in the uses and methods of the invention such as in the treatment and prevention of pain, it is a N-methylpiperazine ring.

The compounds of formulae (1) and (1a) and pharmacologically active prodrugs and salts thereof contain some substituents for which there exist isosteres, and compounds containing such isosteres in place of said substituents also form a part of the present invention. For example, where the compounds of formulae (1) and (1a) and pharmacologically active prodrugs or salts thereof contain a carboxyl group, this can be replaced with a tetrazolyl group.

Hydrates or solvates of the compounds of formulae (1) and (1a), prodrugs thereof and pharmacologically acceptable salts thereof can also be used and form a part of the invention.

Some compounds of formulae (1) and (1a) and their pharmacologically acceptable salts or prodrugs thereof of the present invention may have one or more asymmetric carbons, and optical isomers (including diastereomers) due to the presence of asymmetric carbon atom(s) in the molecule can exist. These isomers are included in the present invention, both as individual isomers and mixtures thereof in all possible ratios.

The compounds of formulae (1) and (1a) of the present invention can form pharmacologically acceptable salts and pro-drugs and these form a part of the present invention. Examples of such salts include inorganic salts such as ammonium salts; organic amine salts such as t-octylamine salts, dibenzylamine salts, morpholine salts, glucosamine salts, phenylglycine alkyl ester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts, N-benzyl-N-phenethylamine salts, piperazine salts, tetramethylammonium salts and tris(hydroxymethyl)aminomethane salts; hydrohalogenated salts such as hydrofluoric acid salts, hydrochlorides, hydrobromides and hydroiodides; inorganic acid salts such as nitrates, perchlorates, sulfates and phosphates; lower alkanesulfonate salts such as methanesulfonates, trifluoromethanesulfonates and ethanesulfonates; arylsulfonate salts such as benzensulfonates and p-toluenesulfonates; organic acid salts such as acetates, malates, fumarates, succinates, citrates, tartrates, oxalates and maleates; and amino acid salts such as ornithinates, glutamates and aspartates. Of these, organic amine salts are more preferred and triethylamine salts are most preferred.

The compounds of formulae (1) and (1a) of the present invention can be administered in the form of prodrugs. Prodrugs are derivatives of the pharmacologically active compound in which one or more of the substituents on said compound are protected by a group which is then removable by a biological process (e.g. hydrolysis) in vivo after administration to the patient. Many suitable prodrugs are well-known to the person in the art and can be found, for example, in “Greene's Protective Groups in Organic Synthesis”, 4^{th} Edition, 2006, Wiley-VCH. Suitable examples of such prodrugs include pharmacologically acceptable esters of the compound having the formulae (1) or (1a) wherein a carboxyl moiety of the compound having the formula (1) or (1a) is esterified. The pharmacologically acceptable esters are not particularly restricted, and can be selected by a person with an ordinary skill in the art. In the case of said esters, it is preferable that such esters can be cleaved by a biological process such as hydrolysis in vivo. The group constituting the said esters (the group shown as R when the esters thereof are expressed as —COOR) can be, for example, a C₁-C₄ alkoxy C₁-C₄ alkyl group such as methoxyethyl, 1-ethoxyethyl, 1-methyl-1-methoxyethyl, 1-(isopropoxy)ethyl, 2-methoxyethyl, 2-ethoxyethyl, 1,1-dimethyl-1-methoxymethyl, ethoxymethyl, propoxymethyl, isopropoxymethyl, butoxymethyl or t-butoxymethyl; a C₁-C₄ alkoxylated C₁-C₄ alkoxy C₁-C₄ alkyl group such as 2-methoxyethoxymethyl; a C₆-C₁₀ aryloxy C₁-C₄ alkyl group such as phenoxymethyl; a halogenated C₁-C₄ alkoxy C₁-C₄ alkyl group such as 2,2,2-trichloroethoxymethyl or bis(2-chloroethoxy)methyl; a C₁-C₄ alkoxycarbonyl C₁-C₄ alkyl group such as methoxycarbonylmethyl; a cyano C₁-C₄ alkyl group such as cyanomethyl or 2-cyanoethyl; a C₁-C₄ alkylthiomethyl group such as methylthiomethyl or ethylthiomethyl; a C₆-C₁₀ arylthiomethyl group such as phenylthiomethyl or naphthylthiomethyl; a C₁-C₄ alkylsulfonyl C₁-C₄ lower alkyl group, which may be optionally substituted with a halogen atom(s) such as 2-methanesulfonylethyl or 2-trifluoromethanesulfonylethyl; a C₆-C₁₀ arylsulfonyl C₁-C₄ alkyl group such as 2-benzenesulfonylethyl or 2-toluenesulfonylethyl; a C₁-C₇ aliphatic acyloxy C₁-C₄ alkyl group such as formyloxymethyl, acetoxymethyl, propionyloxymethyl, butyryloxymethyl, pivaloyloxymethyl, valeryloxymethyl, isovaleryloxymethyl, hexanoyloxymethyl, 1-formyloxyethyl, 1-acetoxyethyl, 1-propionyloxyethyl, 1-butyryloxyethyl, 1-pivaloyloxyethyl, 1-valeryloxyethyl, 1-isovaleryloxyethyl, 1-hexanoyloxyethyl, 2-formyloxyethyl, 2-acetoxyethyl, 2-propionyloxyethyl, 2-butyryloxyethyl, 2-pivaloyloxyethyl, 2-valeryloxyethyl, 2-isovaleryloxyethyl, 2-hexanoyloxyethyl, 1-formyloxypropyl, 1-acetoxypropyl, 1-propionyloxypropyl, 1-butyryloxypropyl, 1-pivaloyloxypropyl, 1-valeryloxypropyl, 1-isovaleryloxypropyl, 1-hexanoyloxypropyl, 1-acetoxybutyl, 1-propionyloxybutyl, 1-butyryloxybutyl, 1-pivaloyloxybutyl, 1-acetoxypentyl, 1-propionyloxypentyl, 1-butyryloxypentyl, 1-pivaloyloxypentyl or 1-pivaloyloxyhexyl; a C₅-C₆ cycloalkylcarbonyloxy C₁-C₄ alkyl group such as cyclopentylcarbonyloxymethyl, cyclohexylcarbonyloxymethyl, 1-cyclopentylcarbonyloxyethyl, 1-cyclohexylcarbonyloxyethyl, 1-cyclopentylcarbonyloxypropyl, 1-cyclohexylcarbonyloxypropyl, 1-cyclopentylcarbonyloxybutyl or 1-cyclohexylcarbonyloxybutyl; a C₆-C₁₀ arylcarbonyloxy C₁-C₄ alkyl group such as benzoyloxymethyl; a C₁-C₆ alkoxycarbonyloxy C₁-C₄ alkyl group such as methoxycarbonyloxymethyl, 1-(methoxycarbonyloxy)ethyl, 1-(methoxycarbonyloxy)propyl, 1-(methoxycarbonyloxy)butyl, 1-(methoxycarbonyloxy)pentyl, 1-(methoxycarbonyloxy)hexyl, ethoxycarbonyloxymethyl, 1-(ethoxycarbonyloxy)ethyl, 1-(ethoxycarbonyloxy)propyl, 1-(ethoxycarbonyloxy)butyl, 1-(ethoxycarbonyloxy)pentyl, 1-(ethoxycarbonyloxy)hexyl, propoxycarbonyloxymethyl, 1-(propoxycarbonyloxy)ethyl, 1-(propoxycarbonyloxy)propyl, 1-(propoxycarbonyloxy)butyl, isopropoxycarbonyloxymethyl, 1-(isopropoxycarbonyloxy)ethyl, 1-(isopropoxycarbonyloxy)butyl, butoxycarbonyloxymethyl, 1-(butoxycarbonyloxy)ethyl, 1-(butoxycarbonyloxy)propyl, 1-(butoxycarbonyloxy)butyl, isobutoxycarbonyloxymethyl, 1-(isobutoxycarbonyloxy)ethyl, 1-(isobutoxycarbonyloxy)propyl, 1-(isobutoxycarbonyloxy)butyl, t-butoxycarbonyloxymethyl, 1-(t-butoxycarbonyloxy)ethyl, pentyloxycarbonyloxymethyl, 1-(pentyloxycarbonyloxy)ethyl, 1-(pentyloxycarbonyloxy)propyl, hexyloxycarbonyloxymethyl, 1-(hexyloxycarbonyloxy)ethyl or 1-(hexyloxycarbonyloxy)propyl; a C₅-C₆ cycloalkyloxycarbonyloxy C₁-C₄ alkyl group such as cyclopentyloxycarbonyloxymethyl, 1-(cyclopentyloxycarbonyloxy)ethyl, 1-(cyclopentyloxycarbonyloxy)propyl, 1-(cyclopentyloxycarbonyloxy)butyl, cyclohexyloxycarbonyloxymethyl, 1-(cyclohexyloxycarbonyloxy)ethyl, 1-(cyclohexyloxycarbonyloxy)propyl or 1-(cyclohexyloxycarbonyloxy)butyl; a [5-(C₁-C₄ alkyl)-2-oxo-1,3-dioxolen-4-yl]methyl group such as (5-methyl-2-oxo-1,3-dioxolen-4-yl)methyl, (5-ethyl-2-oxo-1,3-dioxolen-4-yl)methyl, (5-propyl-2-oxo-1,3-dioxolen-4-yl)methyl, (5-isopropyl-2-oxo-1,3-dioxolen-4-yl)methyl or (5-butyl-2-oxo-1,3-dioxolen-4-yl)methy; a [5-(phenyl, which may be optionally substituted with a C₁-C₄ alkyl, C₁-C₄ alkoxy or halogen atom(s))-2-oxo-1,3-dioxolen-4-yl]methyl group such as (5-phenyl-2-oxo-1,3-dioxolen-4-yl)methyl, [5-(4-methylphenyl)-2-oxo-1,3-dioxolen-4-yl]methyl, [5-(4-methoxyphenyl)-2-oxo-1,3-dioxolen-4-yl]methyl, [5-(4-fluorophenyl)-2-oxo-1,3-dioxolen-4-yl]methyl or [5-(4-chlorophenyl)-2-oxo-1,3-dioxolen-4-yl]methyl; or a phthalidyl group, which may be optionally substituted with a C₁-C₄ alkyl or C₁-C₄ alkoxy group(s), such as phthalidyl, dimethylphthalidyl or dimethoxyphthalidyl, and is preferably a pivaloyloxymethyl group, phthalidyl group or (5-methyl-2-oxo-1,3-dioxolen-4-yl)methyl group, and more preferably a (5-methyl-2-oxo-1,3-dioxolen-4-yl)methyl group.

In the combinations according to the forty-eighth aspect of the present invention, typical examples of each of the classes of compounds that can be used in combination with the compounds having the general formula (1) or (1a) or a pharmacologically acceptable salt or prodrug thereof of the present invention are as follows:

1. Examples of muscarinic receptor antagonists (including but not limited to selective M3 antagonists) include esoxybutynin, oxybutynin [especially the chloride], tolterodine [especially the tartrate], solifenacin [especially the succinate], darifenacin [especially the hydrobromide], temiverine, fesoterodine, imidafenacin and trospium [especially the chloride].
2. Examples of β3 adrenergic receptor agonists include YM-178 and solabegron, KUC-7483.
3. Examples of neurokinin K receptor antagonists (including selective NK-1 antagonists) include cizolirtine and casopitant.
4. Examples of vanilloid VR1 agonists include capsaicin, resiniferatoxin and NDG-8243.
5. Examples of calcium channel α2 δ ligands include gabapentin and pregabalin.
6. Examples of potassium channel activators (including activators of KCNQ, BKCa channels, Kv channels and KATP channels) include KW-7158, NS-8 and retigabine.
7. Examples of calcium channel inhibitors (including Cav2.2 channel inhibitors) include ziconotide and NMED-160.
8. Examples of sodium channel blockers include lidocaine, lamotrigine, VX-409, ralfinamide and carbamazepine.
9. Examples of serotonin and norepinephrine reuptake inhibitors (SNRIs) include duloxetine and venlafaxine
10. Examples of 5-HT antagonists including 5-HT1a antagonists and 5HT3 antagonists.
11. Examples of α-1 adrenoceptor antagonists include tamsulosin.
12. Examples of tricyclic antidepressants include amitriptyline, amoxapine, clomipramine, dosulepin (dothiepin), doxepin, imipramine, lofepramine, nortriptyline, and trimipramine.
13. Examples of N-methyl-D-aspartate (NMDA) receptor antagonists include ketamine, memantine, amantadine, AVP-923, NP-1 and EVT-101.
14. Examples of cannabinoid receptor agonists include GW-1000 (Sativex) and KDS-2000.
15. Anti-convulsants. Examples include lacosamide, carbamazepine, topiramate, oxcarbazepine and levetiracetam
16. Examples of aldose reductase inhibitors include tolrestat, zopolrestat, zenarestat, epalrestat, sorbinil, AS-3201, fidarestat, risarestat, ponalrestat and alrestatin.
17. Examples of opioids (e.g. mu opioid agonists) include fentanyl and tapentadol.
18. Examples of a adrenoceptor agonists include a₁-adrenoceptor agonists such as ethoxamine, phenylephrine, oxymetazoline, tetrahydralazine and xylometazoline and a₂-adrenoceptor agonists such as clonidine, guanabenz, guanfacine and α-methyldopa.
19. Examples of P2X receptor antagonists including P2X2 receptor antagonists and P2X7 receptor antagonists.
20. Examples of acid-sensing ion channel modulators include amiloride.
21. Examples of NGF receptor modulators include trkA.
22. Examples of nicotinic acetylcholine receptor modulators include A-85380, tebanicline, ABT-366833, ABT-202, ABT-894, epibatidine analogs and SIB-1663.
23. Examples of synaptic vesicle protein 2A ligands include brivaracetam.

Examples of the administration form of the combination of the present invention are the same as given above for the compounds of general formula (1) or (1a) and pharmacologically acceptable salts or pro-drugs thereof. The particular form can be chosen depending upon the condition to be treated and the nature of the compounds being administered in combination. For example, a combination of a compound of general formula (I) or (1a) or a pharmacologically acceptable salt or pro-drug thereof with lidocaine could be administered transdermally by means of a patch while a combination with ziconotide could be administered transmucosally.

Examples of the administration form of a compound having general formulae (1) or (1a) of the present invention, or a pharmacologically acceptable salt or pro-drug thereof, include oral administration by tablets, capsules, granules, powders or syrups, and parenteral administration by injection, patches or suppositories. Moreover, compounds having the general formula (1) or (1a) or a pharmacologically acceptable salt or pro-drug thereof of the present invention can also be administered by pulmonary administration in the form of a powder, solution or suspension. Preparations for these administrations are produced by known methods using additives such as excipients, lubricants, binders, disintegrants, stabilizers, corrigents, diluents and so forth.

Examples of excipients include organic excipients such as sugar derivatives, e.g. lactose, sucrose, glucose, mannitol or sorbitol, starch derivatives, e.g. corn starch, potato starch, α-starch, dextrin or carboxymethyl starch, cellulose derivatives, e.g. crystalline cellulose, low substituted hydroxypropyl cellulose, hydroxypropyl methyl cellulose, carboxymethyl cellulose, calcium carboxymethyl cellulose or internally crosslinked sodium carboxymethyl cellulose, and gum Arabic, dextran or pullulan; and, inorganic excipients such as silicate derivatives, e.g. light anhydrous silicic acid, synthetic aluminium silicate or magnesium aluminium metasilicate, phosphates, e.g. calcium phosphate, carbonates, e.g. calcium carbonate, or sulfates, e.g. calcium sulfate.

Examples of lubricants include stearic acid and metal stearates such as calcium stearate or magnesium stearate; talc; colloidal silica; waxes such as bee gum or spermaceti; boric acid; adipic acid; sulfates such as sodium sulfate; glycol; fumaric acid; sodium benzoate; DL-leucine; sodium fatty acid salts; lauryl sulfates such as sodium lauryl sulfate or magnesium lauryl sulfate; silicic acids such as silicic anhydride or silicate hydrate; and, starch derivatives.

Examples of binders include polyvinylpyrrolidone, Macrogol and compounds similar to the aforementioned excipients.

Examples of disintegrants agents include compounds similar to the aforementioned excipients, and chemically cross-linked starches and celluloses such as cross sodium carmellose, sodium carboxymethyl starch or crosslinked polyvinylpyrrolidone.

Examples of stabilizers include paraoxybenzoate esters such as methyl paraben or propyl paraben; alcohols such as chlorobutanol, benzyl alcohol or phenyl ethyl alcohol; benzalkonium chloride; phenols such as phenol or cresol; thimerosal; dehydroacetic acid; and, sorbic acid.

Examples of corrigents include ordinarily used sweeteners, sour flavourings and fragrances.

In the case of producing a solution or suspension for pulmonary administration of a compound having the general formula (1) or general formula (1a) or pharmacologically acceptable salt or pro-drug thereof of the present invention, for example, said solution or suspension can be produced by dissolving or suspending crystals of the present invention in water or in a mixture of water and an auxiliary solvent (e.g., ethanol, propylene glycol or polyethylene glycol). Such a solution or suspension may also contain an antiseptic (e.g., benzalkonium chloride), solubilizing agent (e.g., a polysorbate such as Tween 80 or Span 80 or surface activator such as benzalkonium chloride), buffer, isotonic agent (e.g., sodium chloride), absorption promoter and/or thickener. In addition, the suspension may additionally contain a suspending agent (such as microcrystalline cellulose or sodium carboxymethyl cellulose).

A composition for pulmonary administration produced in the manner described above is administered directly into the nasal cavity or oral cavity by a typical means in the field of inhalants (using, for example, a dropper, pipette, cannula or atomizer). In the case of using an atomizer, crystals of the present invention can be atomized as an aerosol in the form of a pressurized pack together with a suitable nebula (for example, a chlorofluorocarbon such as dichlorofluoromethane, trichlorofluoromethane or dichlorotetrafluoroethane, or a gas such as carbon dioxide), or they can be administered using a nebulizer.

The amount of a compound having the general formula (1) or general formula (1a) or pharmacologically acceptable salt or pro-drug thereof of the present invention used varies depending on the symptoms, age, administration method and so forth, and may be administered either in a single dose or by dividing into multiple doses according to the symptoms.

### Synthesis of the Compounds of the Invention

The analogues of the present invention can be synthesised using standard methods and principles as illustrated in the general schemes below:

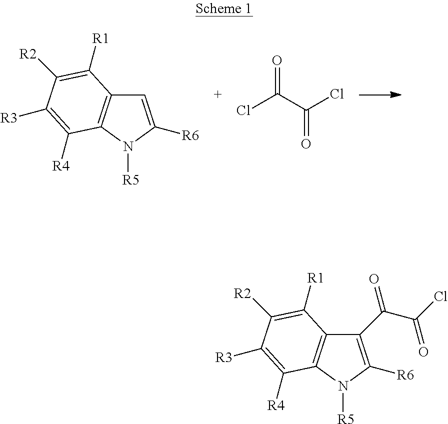

Indole-3-glyoxyl chlorides (2-(1H-indol-3-yl)-2-oxoacetyl chlorides) can be prepared from indoles and oxalyl chloride as shown in Scheme 1. Reaction of indole-3-glyoxyl chlorides with alcohols provide the corresponding esters, while reaction with amines provide the respective amides (Scheme 2).

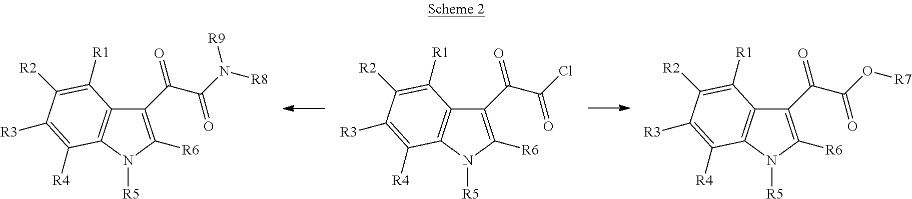

### General Experimental

### LCMS Method A

| Column: | XBridge C18 2 × 30 mm, 5 μm | | |
|---|---|---|---|
| Mobile Phase: | Eluent A: 10 mM Aqueous Ammonium Bicarbonate | | |
| | Eluent B: Acetonitrile | | |
| Gradient: | Time (min) | % A | % B |
| | 0 | 100 | 0 |
| | 0.1 | 100 | 0 |
| | 3.1 | 5 | 95 |
| | 3.9 | 5 | 95 |
| | 4.0 | 100 | 0 |

| | |
|---|---|
| Run time: | 5 min |
| Flow rate: | 1.0 ml/min |
| Injection volume: | 5 μl |
| Column | 25° C. |
| temperature: | |
| Detection: | UV (TAC 215-350 nm), MS (TIC 100-1000 |
| | mz, ESI+ or ESI−) |

### LCMS Method B

| Column: | XBridge C18 2 × 30 mm, 5 μm | | |
|---|---|---|---|
| Mobile Phase: | Eluent A: 0.1% Aqueous Formic Acid | | |
| | Eluent B: Acetonitrile | | |
| Gradient: | Time (min) | % A | % B |
| | 0 | 100 | 0 |
| | 0.1 | 100 | 0 |
| | 3.1 | 5 | 95 |
| | 3.9 | 5 | 95 |
| | 4.0 | 100 | 0 |

| | |
|---|---|
| Run time: | 5 min |
| Flow rate: | 1.0 ml/min |
| Injection volume: | 5 μl |
| Column | 25° C. |
| temperature: | |
| Detection: | UV (TAC 215-350 nm), MS (TIC 100-1000 |
| | mz, ESI+ or ESI−) |

### LCMS Method C

| Column: | XBridge C18 4.6 × 50 mm, 5 μm | | |
|---|---|---|---|
| Mobile Phase: | Eluent A: 0.05% Aqueous Formic Acid | | |
| | Eluent B: 0.05% Formic Acid in Acetonitrile | | |
| Gradient: | Time (min) | % A | % B |
| | 0 | 95 | 5 |
| | 0.5 | 95 | 5 |
| | 13.00 | 5 | 95 |
| | 13.50 | 5 | 95 |
| | 14.50 | 95 | 5 |
| | 15.00 | 95 | 5 |

| | |
|---|---|
| Run time: | 15 min |
| Flow rate: | 1.5 ml/min |
| Injection volume: | 5 μl |
| Column | 30° C. |
| temperature: | |
| Detection: | UV (TAC 210-400 nm), MS (TIC 100-700 |
| | mz, ESI+, ESI−, APCI+, APCI−) |

### GENERAL PROCEDURES

### Example 1

### 2-Oxo-2-(6-trifluoromethoxy-1H-indol-yl)acetic acid (6)

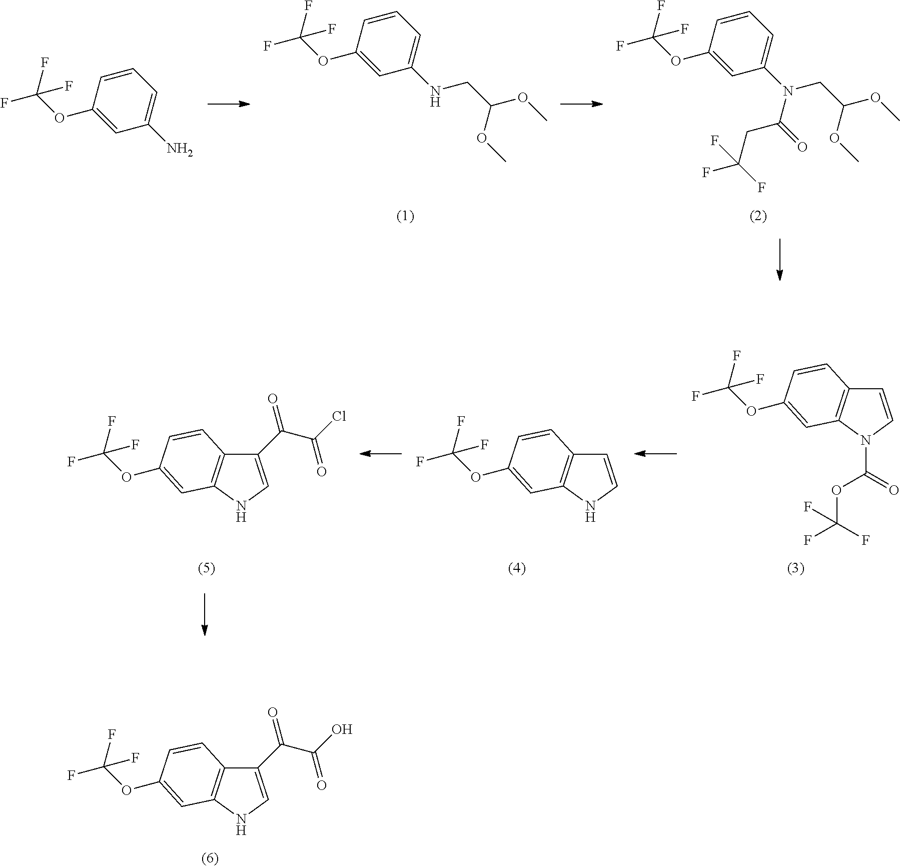

Glyoxyl dimethyl acetal (1.27 g, 12.2 mmol, 2.2 eq) was added to 3-trifluoromethoxyaniline (1.00 g, 5.7 mmol, 1.0 eq) in EtOH (15 ml). Palladium on charcoal (10% w/w; 0.20 g) was added and the reaction was placed under hydrogen (1 atmosphere). The reaction was stirred at room temperature for 16 h. The reaction mixture was filtered through celite and the filtrate was concentrated under reduced pressure. The residue was taken up in brine (20 ml) and the product was extracted with EtOAc (3×20 ml). The combined organic extracts were dried over Na₂SO₄ and concentrated under reduced pressure to provide (1) as a yellow oil (1.43 g, 4.9 mmol, 86%).

Trifluoroacetic anhydride (1.10 ml, 7.8 mmol, 1.1 eq) was added to (1) (2.08 g, 7.1 mmol, 1.0 eq) and triethylamine (1.20 ml, 8.6 mmol, 1.2 eq) in hexane (20 ml) at 0° C. The reaction was allowed to reach room temperature and stirred for 16 h. The solvent was removed under reduced pressure and water (50 ml) was added. The product was extracted with EtOAc (3×50 ml) and the combined organic extracts were dried over Na₂SO₄ and concentrated under reduced pressure. The residue was purified by flash column chromatography on silica using EtOAc and hexane as an eluent to provide (2) (1.38 g, 3.6 mmol, 50%).

Trifluoroacetic anhydride (7 ml) and TFA (15 ml) were added to (2) (1.38 g, 3.6 mmol, 1.0 eq) and the mixture was heated at reflux for 72 h. The solvent was removed under reduced pressure. (3) was isolated by flash column chromatography on silica using EtOAc and hexane. 2 M Aqueous KOH solution (2 ml) was added to a solution of (3) in MeOH (2 ml) at room temperature and the reaction was stirred for 16 h. Water (50 ml) was added and the mixture was extracted with EtOAc (3×50 ml). The combined organic extracts were dried over Na₂SO₄ and concentrated under reduced pressure. The residue was purified by flash column chromatography on silica using EtOAc and hexane as an eluent to provide (4) (40 mg, 0.2 mmol, 6% over two steps).

Oxalyl chloride (0.19 ml, 2.2 mmol, 1.1 eq) was added to (4) (0.40 g, 2.0 mmol, 1.0 eq) in Et₂O (5 ml) at 0° C. The reaction was allowed to reach room temperature and stirred for 4 h after which time the product was isolated by filtration. The filtrate was evaporated to give further product. The product was washed with Et₂O (2×10 ml) to provide (5) as a yellow solid (0.58 mg, 2.0 mmol, 100%).

2 M aqueous NaOH solution (2 ml) was added to (5) (50 mg, 0.17 mmol, 1.0 eq) in tetrahydrofuran (1 ml) and the reaction was stirred at room temperature for 16 h. The layers were separated and the aqueous phase was acidified to pH 1 using 6 M aqueous HCl solution. The product was extracted with EtOAc (3×10 ml) and the combined organic extracts were dried over Na₂SO₄ and concentrated under reduced pressure to provide (6) as a solid (34 mg, 0.12 mmol, 73%). LCMS Method C; RT=5.38 min; MH+ 274.0.

### Example 2

### 2-(6-Hydroxymethyl-1H-indol-3-yl)-2-oxoacedc acid (9)

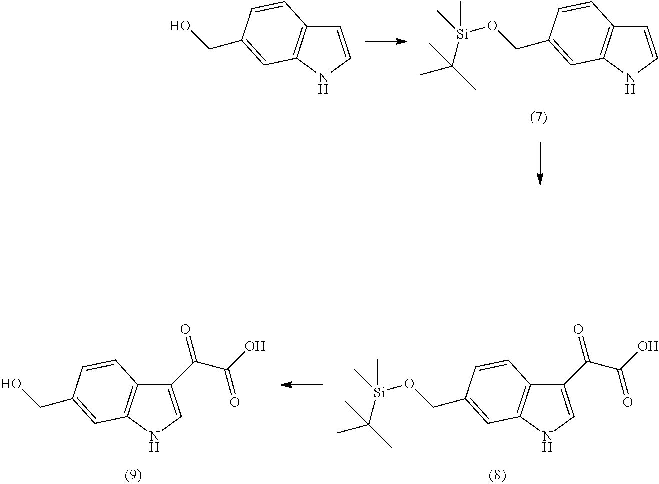

tert-Butyldimethylsilyl chloride (2.20 g, 14.6 mmol, 1.2 eq) was added to a solution of 6-hydroxymethylindole (1.78 g, 12.1 mmol, 1.0 eq) and triethylamine (5.00 ml, 36.3 mmol, 3.0 eq) in CH₂Cl₂ (20 ml) at 0° C. The reaction was allowed to reach room temperature and stirred for 16 h. Water (50 ml) was added and the product was extracted with CH₂Cl₂ (3×50 ml). The combined organic extracts were dried over Na₂SO₄ and concentrated under reduced pressure. The residue was purified by flash column chromatography on silica using EtOAc and hexane as an eluent to provide (7) off-white solid (2.85 g, 10.9 mmol, 90%).

Oxalyl chloride (1.00 ml, 11.6 mmol, 1.1 eq) was added to (7) (2.85 g, 10.9 mmol, 1.0 eq) in Et₂O (10 ml) at 0° C. The reaction was allowed to reach room temperature and stirred for 4 h after which time the precipitate was isolated by filtration. The solid was dissolved in 2 M aqueous NaOH solution (20 ml) and the reaction mixture was stirred at room temperature for 16 h. The basic solution was washed with Et₂O (20 ml) and acidified to pH 1 using 6 M aqueous HCl solution. The product was extracted with EtOAc (3×100 ml) and the combined organic extracts were dried over Na₂SO₄ and concentrated under reduced pressure to provide (8) as a yellow solid (3.53 g, 10.6 mmol, 97%).

Tetrabutylammonium fluoride (15.0 ml of a 1.0 M solution in tetrahydrofuran, 15.0 mmol, 5.0 eq) was added to (8) (1.00 g, 3.0 mmol, 1.0 eq) in tetrahydrofuran (10 ml) at 0° C. The reaction was allowed to reach room temperature and stirred for 4 h. The solvent was removed under reduced pressure and 2 M aqueous NaOH solution was added until a pH 10-12 was reached. The basic solution was washed with CH₂Cl₂ (2×50 ml) and acidified to pH 1 using 6 M aqueous HCl solution. The precipitate was isolated by filtration, washed with EtOAc (10 ml) and hexane (10 ml) and dried under reduced pressure at 60° C. (9) was isolated as a light brown solid (574 mg, 2.6 mmol, 87%). LCMS Method A; RT=0.22 min; MH+ 220.0.

General Procedure for the Synthesis of Indole-3-glyoxylic Acids

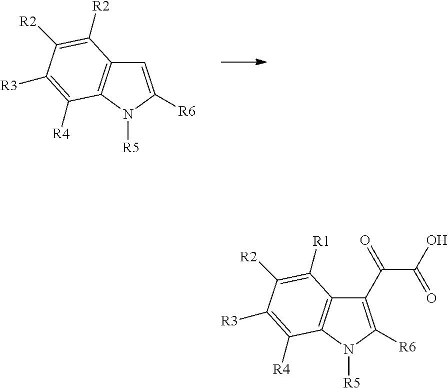

Method A: Oxalyl chloride (1.0 eq) was added to the required indole (1.0 eq) in Et₂O (1.5 ml/mmol) at 0° C. The reaction was allowed to reach room temperature and stirred for 4 h. After cooling to 0° C. saturated aqueous NaHCO₃ solution (1.5 ml/mmol) was added slowly and the reaction mixture was allowed to reach room temperature and stirred for 16 h. The layers were separated and the aqueous phase was acidified to pH 1 using 6 M aqueous HCl solution. The product was extracted with EtOAc (3×20 ml/mmol) and the combined organic extracts were dried over Na₂SO₄ and concentrated under reduced pressure to provide the desired indole-3-glyoxylic acid. If required the product was purified by trituration with an appropriate solvent.

Method B: Oxalyl chloride (1.0 eq) was added to the required indole (1.0 eq) in Et₂O (1.5 ml/mmol) at 0° C. The reaction was allowed to reach room temperature and stirred for 4 h after which time the precipitate was isolated by filtration. The solid was dissolved in 2 M aqueous NaOH solution (1.5 ml/mmol) and the reaction mixture was stirred at room temperature for 16 h. The basic solution was washed with Et₂O (1.5 ml/mmol) and acidified to pH 1 using 6 M aqueous HCl solution. The product was extracted with EtOAc (3×20 ml/mmol) and the combined organic extracts were dried over Na₂SO₄ and concentrated under reduced pressure to provide the desired indole-3-glyoxylic acid. If required the product was purified by trituration with an appropriate solvent.

General Procedure for the Synthesis of Indole-3-glyoxyl Chlorides

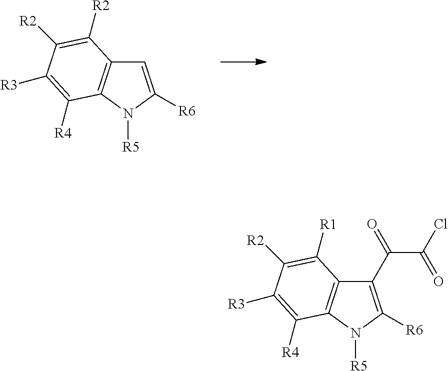

Oxalyl chloride (1.0 eq) was added to the required indole (1.0 eq) in Et₂O (1.5 ml/mmol) at 0° C. The reaction was allowed to reach room temperature and stirred for 4 h after which time the desired indole-3-glyoxyl chloride was either isolated by filtration or by concentration of the reaction mixture followed by trituration of the residue with Et₂O.

### Example 3

### (2-morpholin-4-ylethyl)2-(6-methoxy-1H-indol-3-yl)-2-oxoacetate (10)

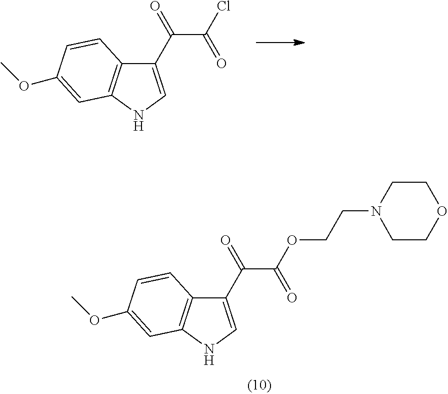

N-(2-Hydroxyethyl)morpholine (420 mg, 3.2 mmol, 1.2 eq) and catalytic N-dimethylaminopyridine were added to 6-methoxyindole-3-glyoxyl chloride (642 mg, 2.7 mmol, 1.0 eq) in toluene (10 ml) and the reaction mixture was heated to 70° C. for 18 h. The solvent was removed under reduced pressure and the residue was purified by flash chromatography on silica using CH₂Cl₂ and MeOH as an eluent followed by trituration with MeOH to provide (10) (10 mg, 0.03 mmol, 1%). LCMS Method C; RT=4.14 min; MH+ 333.1.

### Example 4

### (2-methanesulfonylethyl)2-(6-methoxy-1H-indol-3-yl)-2-oxoacetate (11)

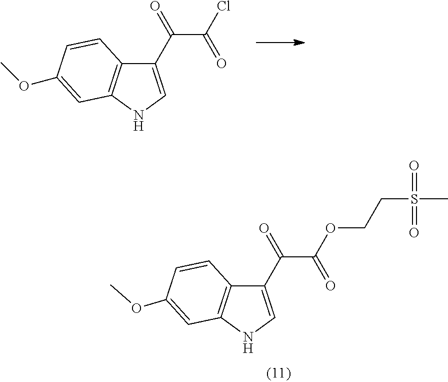

2-(Methylsulfonyl)ethanol (400 mg, 3.2 mmol, 1.2 eq) and triethylamine (0.75 ml, 5.4 mmol, 2.0 eq) were added to 6-methoxyindole-3-glyoxyl chloride (642 mg, 2.7 mmol, 1.0 eq) in CH₂Cl₂ (10 ml) at room temperature and the reaction mixture was stirred for 16 h. The solvent was removed under reduced pressure and the residue was triturated with CH₂Cl₂ to provide (11) (228 mg, 0.70 mmol, 26%). LCMS Method C; RT=2.83 min; MH+ 326.0.

General Procedure for the Synthesis of Indole-3-glyoxylic Acid Ethyl Esters

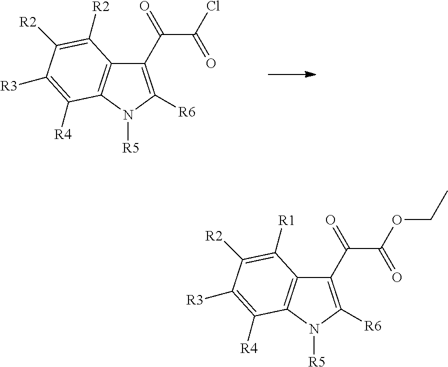

Method C: Absolute ethanol (5 ml/mmol) was added to the required indole-3-glyoxyl chloride (1.0 eq) at room temperature and the reaction mixture was stirred for 48 h. In cases where precipitation of product occurred, this was isolated by filtration. Otherwise the solvent was removed under reduced pressure and the residue was dissolved in EtOAc (20 ml/mmol). The organic solution was washed with saturated aqueous NaHCO₃ solution (2×20 ml/mmol), dried over Na₂SO₄ and concentrated under reduced pressure to provide the desired ethyl ester. If required the product was purified by either trituration with an appropriate solvent, flash column chromatography or by preparative HPLC.

### Example 5

### 1-(6-hydroxy-1H-indol-3-yl)-2-morpholinoethane-1,2-dione (13)

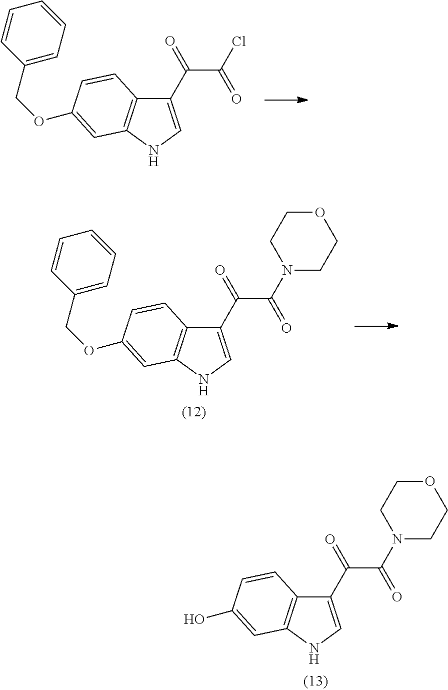

Morpholine (0.28 ml, 3.2 mmol, 4.0 eq) was added to 6-benzyloxyindole-3-glyoxyl chloride (0.25 mg, 0.80 mmol, 1.0 eq) in tetrahydrofuran (6 ml) at 0° C. and the reaction was allowed to reach room temperature and stirred for 4 h. The solvent was removed under reduced pressure and the residue was dissolved in EtOAc (50 ml). The organic solution was washed with 10% aqueous citric acid solution (25 ml), saturated aqueous NaHCO₃ solution (25 ml), dried over MgSO₄ and concentrated under reduced pressure. The residue was purified by flash column chromatography using CH₂Cl₂ and MeOH as an eluent to provide the (12) (0.24 g, 0.66 mmol, 83%)

Palladium on charcoal (10% w/w; 0.03 g) was added to (12) (0.24 g, 0.66 mmol, 1.0 eq) in tetrahydrofuran (15 ml). The reaction was placed under hydrogen (1 atmosphere) and stirred at room temperature for 16 h. The reaction mixture was filtered through celite and the filtrate was concentrated under reduced pressure. The residue was triturated with EtOAc to provide (13) (0.12 g, 0.44, 67%). LCMS Method A; RT=1.25 min; MH+ 275.1.

### Example 6

### 1-(6-Hydroxy-1H-indol-3-yl)-2-(4-methylpiperazin-1-yl)ethane-1,2-dione (15)

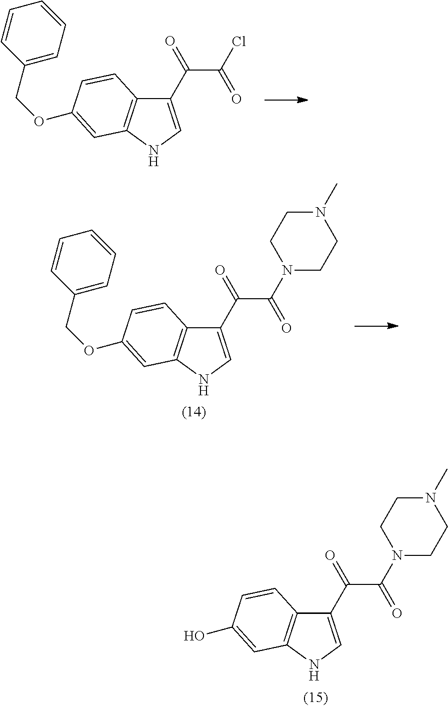

N-Methylpiperazine (0.32 ml, 3.0 mmol, 3.0 eq) was added to 6-benzyloxyindole-3-glyoxyl chloride (0.30 mg, 1.0 mmol, 1.0 eq) in tetrahydrofuran (6 ml) at 0° C. and the reaction was allowed to reach room temperature and stirred for 4 h. The solvent was removed under reduced pressure and the residue was dissolved in EtOAc (50 ml). The organic solution was washed with saturated aqueous NaHCO₃ solution (25 ml) and brine (25 ml), dried over MgSO₄ and concentrated under reduced pressure. The residue was purified by flash column chromatography using CH₂Cl₂ and MeOH as an eluent to provide the (14) (0.29 g, 0.77 mmol, 77%)

Palladium on charcoal (10% w/w; 0.03 g) was added to (14) (0.29 g, 0.77 mmol, 1.0 eq) in MeOH (20 ml). The reaction was placed under hydrogen (1 atmosphere) and stirred at room temperature for 16 h. The reaction mixture was filtered through celite and the filtrate was concentrated under reduced pressure. The residue was purified by flash column chromatography using CH₂Cl₂ and MeOH as an eluent to provide the (15) (0.10 g, 0.35, 45%). LCMS Method A; RT=1.25 min; MH+ 288.1.

### Example 7

### 1-(6-Methoxy-1H-indol-3-yl)-2-piperazin-1-yl-ethane-1,2-dione (17)

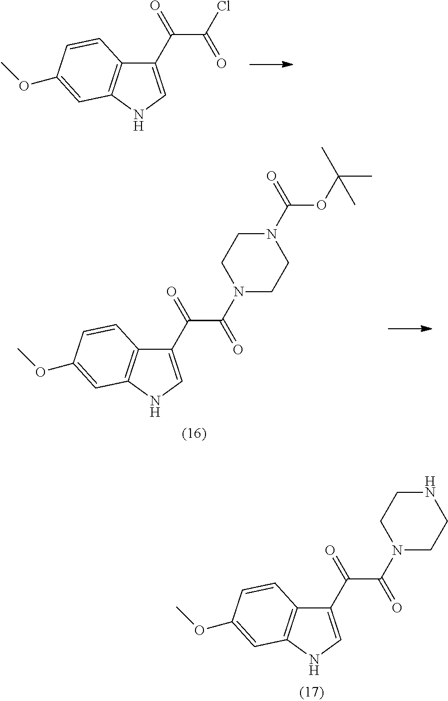

N-(tert-Butoxycarbonyl)piperazine (745 mg, 4.0 mmol, 1.5 eq) and triethylamine (0.75 ml, 5.4 mmol, 2.0 eq) were added to 6-methoxyindole-3-glyoxyl chloride (642 mg, 2.7 mmol, 1.0 eq) in CH₂Cl₂ (10 ml) at 0° C. The reaction mixture was allowed to reach room temperature and stirred for 16 h. The solvent was removed under reduced pressure and the residue was purified by flash column chromatography eluting with CH₂Cl₂ and MeOH to provide (16). 4 M HCl solution in dioxane (10 ml) was added to a solution of (16) in MeOH (5 ml) and the reaction mixture was stirred at room temperature for 16 h. The reaction mixture was concentrated under reduced pressure and the residue was purified by preparative HPLC to provide (17) (47 mg, 0.16 mmol, 6% over two steps). LCMS Method C; RT=3.20 min; MH+ 275.1.

General Procedure for the Synthesis of Indole-3-glyoxyamides

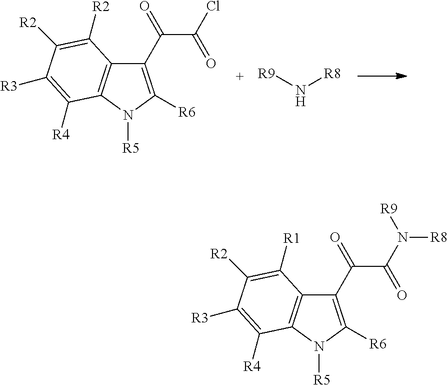

Method D: The required amine (1.0-5.0 eq) and, if necessary, a tertiary base (2.0-3.0 eq) were added to the required indole-3-glyoxyl chloride (1.0 eq) in tetrahydrofuran (5 ml/mmol) at 0° C. and the reaction was allowed to reach room temperature and stirred for 16 h. The solvent was removed under reduced pressure, and the residue was dissolved in EtOAc (20 ml/mmol). The organic solution was washed with, where appropriate, 2 M aqueous HCl solution (2×20 ml/mmol) and saturated aqueous NaHCO₃ solution (2×20 ml/mmol), dried over Na₂SO₄ and concentrated under reduced pressure to provide the desired amide. If required the product was purified by either trituration with an appropriate solvent, flash column chromatography or by preparative HPLC.

Method E: The required amine (1.0-1.5 eq) and saturated aqueous NaHCO₃ solution (30 ml/mmol) were added to a suspension of the required indole-3-glyoxyl chloride (1.0 eq) in toluene (30 ml/mmol) at 0° C. and the reaction mixture was allowed to reach room temperature and stirred vigorously for 16 h. The phases were separated and the aqueous phase was extracted with EtOAc (3×30 ml/mmol). The combined organic layers were washed with brine (30 ml/mmol), dried over MgSO₄ and concentrated under reduced pressure to provide the desired amide. If required the product was purified by either trituration with an appropriate solvent, flash column chromatography or by preparative HPLC.

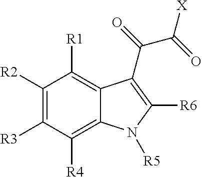

| | | | | | | | | | General | Yield | LCMS | RT | Mass Ion |
|---|---|---|---|---|---|---|---|---|---|---|---|---|---|
| Compound | Name | R1 | R2 | R3 | R4 | R5 | R6 | X | Procedure | (%) | Method | (min) | (MH+) |
| 18 | 2-(4-fluoro-1H-indol- | F | H | H | H | H | H | OH | B | 73 | B | 1.43 | 208.0 |
| | 3-yl)-2-oxoacetic acid | | | | | | | | | | | | |
| 19 | 2-(5-fluoro-1H-indol- | H | F | H | H | H | H | OH | A | 65 | B | 1.79 | 208.0 |
| | 3-yl)-2-oxoacetic acid | | | | | | | | | | | | |
| 20 | 2-(6-fluoro-1H-indol- | H | H | F | H | H | H | OH | A | 51 | B | 1.81 | 208.0 |
| | 3-yl)-2-oxoacetic acid | | | | | | | | | | | | |
| 21 | 2-(7-fluoro-1H-indol- | H | H | H | F | H | H | OH | A | 67 | B | 1.70 | 208.0 |
| | 3-yl)-2-oxoacetic acid | | | | | | | | | | | | |
| 22 | 2-(6-chloro-1H-indol- | H | H | Cl | H | H | H | OH | B | 74 | B | 2.02 | 224.0 |
| | 3-yl)-2-oxoacetic acid | | | | | | | | | | | | |
| 23 | 2-(5-bromo-1H-indol- | H | Br | H | H | H | H | OH | A | 79 | B | 2.10 | 268.0 |
| | 3-yl)-2-oxoacetic acid | | | | | | | | | | | | |
| 24 | 2-(6-bromo-1H-indol- | H | H | Br | H | H | H | OH | B | 80 | B | 2.07 | 269.9 |
| | 3-yl)-2-oxoacetic acid | | | | | | | | | | | | |
| 25 | 2-(1-methyl-1H-indol- | H | H | H | H | Me | H | OH | A | 60 | B | 1.81 | 204.1 |
| | 3-yl)-2-oxoacetic acid | | | | | | | | | | | | |
| 26 | 2-(6-methyl-1H-indol- | H | H | Me | H | H | H | OH | B | 98 | C | 3.88 | 204.1 |
| | 3-yl)-2-oxoacetic acid | | | | | | | | | | | | |
| 27 | 2-(2-methyl-1H-indol- | H | H | H | H | H | Me | OH | A | 35 | C | 2.24 | 204.1 |
| | 3-yl)-2-oxoacetic acid | | | | | | | | | | | | |
| 28 | 2-(6-isopropyl-1H- | H | H | iPr | H | H | H | OH | B | 73 | B | 2.17 | 232.1 |
| | indol-3-yl)-2-oxoacetic | | | | | | | | | | | | |
| | acid | | | | | | | | | | | | |
| 29 | 2-oxo-2-(6- | H | H | CF3 | H | H | H | OH | B | 57 | B | 2.19 | 257.9 |
| | (trifluoromethyl)-1H- | | | | | | | | | | | | |
| | indol-3-yl)acetic acid | | | | | | | | | | | | |
| 30 | 2-(5-methoxy-1H- | H | OMe | H | H | H | H | OH | B | 97 | C | 3.05 | 220.1 |
| | indol-3-yl)-2-oxoacetic | | | | | | | | | | | | |
| | acid | | | | | | | | | | | | |
| 31 | 2-(6-methoxy-1H- | H | H | OMe | H | H | H | OH | B | 57 | A | 1.06 | 220.1 |
| | indol-3-yl)-2-oxoacetic | | | | | | | | | | | | |
| | acid | | | | | | | | | | | | |
| 32 | 2-(5,6-dimethoxy-1H- | H | OMe | OMe | H | H | H | OH | A | 88 | B | 1.56 | 250.0 |
| | indol-3-yl)-2-oxoacetic | | | | | | | | | | | | |
| | acid | | | | | | | | | | | | |
| 33 | 2-(6-ethoxy-1H-indol- | H | H | OEt | H | H | H | OH | B | 91 | A | 1.38 | 234.0 |
| | 3-yl)-2-oxoacetic acid | | | | | | | | | | | | |
| 34 | 2-(6-hydroxy-1H- | H | H | OH | H | H | H | OH | A | 88 | C | 1.23 | 206.1 |
| | indol-3-yl)-2-oxoacetic | | | | | | | | | | | | |
| | acid | | | | | | | | | | | | |
| 35 | 2-(6-cyano-1H-indol- | H | H | CN | H | H | H | OH | B | 43 | B | 1.73 | 215.0 |
| | 3-yl)-2-oxoacetic acid | | | | | | | | | | | | |
| 36 | 2-(6-(methylsulfonyl)- | H | H | SO2Me | H | H | H | OH | B | 70 | B | 1.45 | 267.9 |
| | 1H-indol-3-yl)-2- | | | | | | | | | | | | |
| | oxoacetic acid | | | | | | | | | | | | |
| 37 | ethyl 2-(1H-indol-3- | H | H | H | H | H | H | OEt | C | 37 | A | 1.94 | 218.1 |
| | yl)-2-oxoacetate | | | | | | | | | | | | |
| 38 | ethyl 2-(6-fluoro-1H- | H | H | F | H | H | H | OEt | C | 33 | A | 2.06 | 236.1 |
| | indol-3-yl)-2- | | | | | | | | | | | | |
| | oxoacetate | | | | | | | | | | | | |
| 39 | ethyl 2-oxo-2-(6- | H | H | CF3 | H | H | H | OEt | C | 40 | A | 2.57 | 286.0 |
| | (trifluoromethyl)-1H- | | | | | | | | | | | | |
| | indol-3-yl)acetate | | | | | | | | | | | | |
| 40 | ethyl 2-(6-methoxy- | H | H | OMe | H | H | H | OEt | C | 6 | C | 5.64 | 231.2 |
| | 1H-indol-3-yl)-2- | | | | | | | | | | | | |
| | oxoacetate | | | | | | | | | | | | |
| 41 | ethyl 2-oxo-2-(6- | H | H | OCF3 | H | H | H | OEt | C | 87 | A | 2.60 | 302.0 |
| | (trifluoromethoxy)-1H- | | | | | | | | | | | | |
| | indol-3-yl)acetate | | | | | | | | | | | | |
| 42 | 1-(4-hydroxypiperidin- | H | H | OMe | H | H | H | 4-hydroxy- | E | 7 | C | 3.81 | 303.1 |
| | 1-yl)-2-(6-methoxy- | | | | | | | piperidine | | | | | |
| | 1H-indol-3-yl)-ethane- | | | | | | | | | | | | |
| | 1,2-dione | | | | | | | | | | | | |
| 43 | 1-(1H-indol-3-yl)-2- | H | H | H | H | H | H | morpholine | D | 58 | A | 1.63 | 259.1 |
| | morpholinoethane- | | | | | | | | | | | | |
| | 1,2-dione | | | | | | | | | | | | |
| 44 | 1-(4-fluoro-1H-indol- | F | H | H | H | H | H | morpholine | D | 42 | A | 1.74 | 277.0 |
| | 3-yl)-2- | | | | | | | | | | | | |
| | morpholinoethane- | | | | | | | | | | | | |
| | 1,2-dione | | | | | | | | | | | | |
| 45 | 1-(5-fluoro-1H-indol- | H | F | H | H | H | H | morpholine | D | 61 | A | 1.71 | 277.1 |
| | 3-yl)-2- | | | | | | | | | | | | |
| | morpholinoethane- | | | | | | | | | | | | |
| | 1,2-dione | | | | | | | | | | | | |
| 46 | 1-(6-fluoro-1H-indol- | H | H | F | H | H | H | morpholine | D | 68 | A | 1.73 | 277.1 |
| | 3-yl)-2- | | | | | | | | | | | | |
| | morpholinoethane- | | | | | | | | | | | | |
| | 1,2-dione | | | | | | | | | | | | |
| 47 | 1-(7-fluoro-1H-indol- | H | H | H | F | H | H | morpholine | D | 85 | A | 1.67 | 277.2 |
| | 3-yl)-2- | | | | | | | | | | | | |
| | morpholinoethane- | | | | | | | | | | | | |
| | 1,2-dione | | | | | | | | | | | | |
| 48 | 1-(6-chloro-1H-indol- | H | H | Cl | H | H | H | morpholine | D | 81 | A | 2.06 | 293.0 |
| | 3-yl)-2- | | | | | | | | | | | | |
| | morpholinoethane- | | | | | | | | | | | | |
| | 1,2-dione | | | | | | | | | | | | |
| 49 | 1-(5-bromo-1H-indol- | H | Br | H | H | H | H | morpholine | D | 53 | A | 1.91 | 337.0 |
| | 3-yl)-2- | | | | | | | | | | | | |
| | morpholinoethane- | | | | | | | | | | | | |
| | 1,2-dione | | | | | | | | | | | | |
| 50 | 1-(1-methyl-1H-indol- | H | H | H | H | Me | H | morpholine | D | 53 | A | 1.92 | 273.0 |
| | 3-yl)-2- | | | | | | | | | | | | |
| | morpholinoethane- | | | | | | | | | | | | |
| | 1,2-dione | | | | | | | | | | | | |
| 51 | 1-(6-methyl-1H-indol- | H | H | Me | H | H | H | morpholine | D | 68 | A | 1.96 | 273.0 |
| | 3-yl)-2- | | | | | | | | | | | | |
| | morpholinoethane- | | | | | | | | | | | | |
| | 1,2-dione | | | | | | | | | | | | |
| 52 | 1-(2-methyl-1H-indol- | H | H | H | H | H | Me | morpholine | D | 57 | A | 1.59 | 273.3 |
| | 3-yl)-2- | | | | | | | | | | | | |
| | morpholinoethane- | | | | | | | | | | | | |
| | 1,2-dione | | | | | | | | | | | | |
| 53 | 1-(6-ethyl-1H-indol-3- | H | H | Et | H | H | H | morpholine | D | 42 | A | 1.91 | 288.2 |
| | yl)-2- | | | | | | | | | | | | |
| | morpholinoethane- | | | | | | | | | | | | |
| | 1,2-dione | | | | | | | | | | | | |
| 54 | 1-(6-isopropyl-1H- | H | H | iPr | H | H | H | morpholine | D | 77 | A | 2.04 | 301.3 |
| | indol-3-yl)-2- | | | | | | | | | | | | |
| | morpholinoethane- | | | | | | | | | | | | |
| | 1,2-dione | | | | | | | | | | | | |
| 55 | 1-morpholino-2-(6- | H | H | CF3 | H | H | H | morpholine | D | 17 | A | 2.01 | 368.1 |
| | (trifluoromethyl)-1H- | | | | | | | | | | | | |
| | indol-3-yl)ethane-1,2- | | | | | | | | | | | | |
| | dione | | | | | | | | | | | | |
| 56 | 1-(5-methoxy-1H- | H | OMe | H | H | H | H | morpholine | D | 42 | A | 1.80 | 289.0 |
| | indol-3-yl)-2- | | | | | | | | | | | | |
| | morpholinoethane- | | | | | | | | | | | | |
| | 1,2-dione | | | | | | | | | | | | |
| 57 | 1-(6-methoxy-1H- | H | H | OMe | H | H | H | morpholine | E | 28 | C | 4.29 | 289.0 |
| | indol-3-yl)-2- | | | | | | | | | | | | |
| | morpholinoethane- | | | | | | | | | | | | |
| | 1,2-dione | | | | | | | | | | | | |
| 58 | 1-(6-ethoxy-1H-indol- | H | H | OEt | H | H | H | morpholine | D | 23 | A | 1.78 | 303.1 |
| | 3-yl)-2- | | | | | | | | | | | | |
| | morpholinoethane- | | | | | | | | | | | | |
| | 1,2-dione | | | | | | | | | | | | |
| 59 | 1-(5,6-dimethoxy-1H- | H | OMe | OMe | H | H | H | morpholine | D | 49 | A | 1.49 | 319.1 |
| | indol-3-yl)-2- | | | | | | | | | | | | |
| | morpholinoethane- | | | | | | | | | | | | |
| | 1,2-dione | | | | | | | | | | | | |
| 60 | 1-(5H- | H | OCH2O | OCH2O | H | H | H | morpholine | D | 28 | A | 1.62 | 303.1 |
| | [1,3]dioxolo[4,5- | | | | | | | | | | | | |
| | f]indol-7-yl)-2- | | | | | | | | | | | | |
| | morpholinoethane- | | | | | | | | | | | | |
| | 1,2-dione | | | | | | | | | | | | |
| 61 | 1-morpholino-2-(6- | H | H | OCF3 | H | H | H | morpholine | D | 97 | A | 2.08 | 343.1 |
| | (trifluoromethoxy)-1H- | | | | | | | | | | | | |
| | indol-3-yl)ethane-1,2- | | | | | | | | | | | | |
| | dione | | | | | | | | | | | | |
| 62 | 1-(6-(methylsulfonyl)- | H | H | SO2Me | H | H | H | morpholine | D | 58 | A | 1.42 | 337.1 |
| | 1H-indol-3-yl)-2- | | | | | | | | | | | | |
| | morpholinoethane- | | | | | | | | | | | | |
| | 1,2-dione | | | | | | | | | | | | |
| 63 | 1-(1H-indol-3-yl)-2-(4- | H | H | H | H | H | H | NMe-piperazine | D | 57 | A | 1.77 | 272.0 |
| | methylpiperazin-1- | | | | | | | | | | | | |
| | yl)ethane-1,2-dione | | | | | | | | | | | | |
| 64 | 1-(4-fluoro-1H-indol- | F | H | H | H | H | N | NMe-piperazine | D | 46 | A | 1.72 | 290.0 |
| | 3-yl)-2-(4- | | | | | | | | | | | | |
| | methylpiperazin-1- | | | | | | | | | | | | |
| | yl)ethane-1,2-dione | | | | | | | | | | | | |
| 65 | 1-(5-fluoro-1H-indol- | H | F | H | H | H | H | NMe-piperazine | D | 52 | A | 1.66 | 290.1 |
| | 3-yl)-2-(4- | | | | | | | | | | | | |
| | methylpiperazin-1- | | | | | | | | | | | | |
| | yl)ethane-1,2-dione | | | | | | | | | | | | |
| 66 | 1-(6-fluoro-1H-indol- | H | H | F | H | H | H | NMe-piperazine | D | 71 | A | 1.86 | 290.0 |
| | 3-yl)-2-(4- | | | | | | | | | | | | |
| | methylpiperazin-1- | | | | | | | | | | | | |
| | yl)ethane-1,2-dione | | | | | | | | | | | | |
| 67 | 1-(7-fluoro-1H-indol- | H | H | H | F | H | H | NMe-piperazine | D | 45 | A | 1.63 | 290.3 |
| | 3-yl)-2-(4- | | | | | | | | | | | | |
| | methylpiperazin-1- | | | | | | | | | | | | |
| | yl)ethane-1,2-dione | | | | | | | | | | | | |
| 68 | 1-(6-chloro-1H-indol- | H | H | Cl | H | H | H | NMe-piperazine | D | 46 | A | 2.01 | 306.0 |
| | 3-yl)-2-(4- | | | | | | | | | | | | |
| | methylpiperazin-1- | | | | | | | | | | | | |
| | yl)ethane-1,2-dione | | | | | | | | | | | | |
| 69 | 1-(5-bromo-1H-indol- | H | Br | H | H | H | H | NMe-piperazine | D | 48 | A | 1.84 | 352.0 |
| | 3-yl)-2-(4- | | | | | | | | | | | | |
| | methylpiperazin-1- | | | | | | | | | | | | |
| | yl)ethane-1,2-dione | | | | | | | | | | | | |
| 70 | 1-(1-methyl-1H-indol- | H | H | H | H | Me | H | NMe-piperazine | D | 98 | A | 1.87 | 286.1 |
| | 3-yl)-2-(4- | | | | | | | | | | | | |
| | methylpiperazin-1- | | | | | | | | | | | | |
| | yl)ethane-1,2-dione | | | | | | | | | | | | |
| 71 | 1-(6-methyl-1H-indol- | H | H | Me | H | H | H | NMe-piperazine | D | 74 | A | 1.92 | 286.1 |
| | 3-yl)-2-(4- | | | | | | | | | | | | |
| | methylpiperazin-1- | | | | | | | | | | | | |
| | yl)ethane-1,2-dione | | | | | | | | | | | | |
| 72 | 1-(2-methyl-1H-indol- | H | H | H | H | H | Me | NMe-piperazine | D | 22 | A | 1.57 | 286.1 |
| | 3-yl)-2-(4- | | | | | | | | | | | | |
| | methylpiperazin-1- | | | | | | | | | | | | |
| | yl)ethane-1,2-dione | | | | | | | | | | | | |
| 73 | 1-(6-ethyl-1H-indol-3- | H | H | Et | H | H | H | NMe-piperazine | D | 52 | A | 1.85 | 300.2 |
| | yl)-2-(4- | | | | | | | | | | | | |
| | methylpiperazin-1- | | | | | | | | | | | | |
| | yl)ethane-1,2-dione | | | | | | | | | | | | |
| 74 | 1-(6-isopropyl-1H- | H | H | iPr | H | H | H | NMe-piperazine | D | 75 | A | 1.97 | 314.3 |
| | indol-3-yl)-2-(4- | | | | | | | | | | | | |
| | methylpiperazin-1- | | | | | | | | | | | | |
| | yl)ethane-1,2-dione | | | | | | | | | | | | |
| 75 | 1-(4-methylpiperazin- | H | H | CF3 | H | H | H | NMe-piperazine | D | 7 | A | 2.05 | 340.0 |
| | 1-yl)-2-(6- | | | | | | | | | | | | |
| | (trifluoromethyl)-1H- | | | | | | | | | | | | |
| | indol-3-yl)ethane-1,2- | | | | | | | | | | | | |
| | dione | | | | | | | | | | | | |
| 76 | 1-(5-methoxy-1H- | H | OMe | H | H | H | H | NMe-piperazine | D | 42 | A | 1.58 | 302.1 |
| | indol-3-yl)-2-(4- | | | | | | | | | | | | |
| | methylpiperazin-1- | | | | | | | | | | | | |
| | yl)ethane-1,2-dione | | | | | | | | | | | | |
| 77 | 1-(6-methoxy-1H- | H | H | OMe | H | H | H | NMe-piperazine | E | 33 | C | 3.18 | 302.1 |
| | indol-3-yl)-2-(4- | | | | | | | | | | | | |
| | methylpiperazin-1- | | | | | | | | | | | | |
| | yl)ethane-1,2-dione | | | | | | | | | | | | |
| 78 | 1-(6-ethoxy-1H-indol- | H | H | OEt | H | H | H | NMe-piperazine | D | 10 | A | 1.73 | 316.1 |
| | 3-yl)-2-(4- | | | | | | | | | | | | |
| | methylpiperazin-1- | | | | | | | | | | | | |
| | yl)ethane-1,2-dione | | | | | | | | | | | | |
| 79 | 1-(5,6-dimethoxy-1H- | H | OMe | OMe | H | H | H | NMe-piperazine | D | 50 | A | 1.46 | 332.2 |
| | indol-3-yl)-2-(4- | | | | | | | | | | | | |
| | methylpiperazin-1- | | | | | | | | | | | | |
| | yl)ethane-1,2-dione | | | | | | | | | | | | |
| 80 | 1-(5H- | H | OCH2O | OCH2O | H | H | H | NMe-piperazine | D | 22 | A | 1.58 | 316.1 |
| | [1,3]dioxolo[4,5- | | | | | | | | | | | | |
| | f]indol-7-yl)-2-(4- | | | | | | | | | | | | |
| | methylpiperazin-1- | | | | | | | | | | | | |
| | yl)ethane-1,2-dione | | | | | | | | | | | | |
| 81 | 1-(4-methylpiperazin- | H | H | OCF3 | H | H | H | NMe-piperazine | D | 64 | A | 2.20 | 356.1 |
| | 1-yl)-2-(6- | | | | | | | | | | | | |
| | (trifluoromethoxy)-1H- | | | | | | | | | | | | |
| | indol-3-yl)ethane-1,2- | | | | | | | | | | | | |
| | dione | | | | | | | | | | | | |
| 82 | 1-(4-methylpiperazin- | H | H | SO2Me | H | H | H | NMe-piperazine | D | 43 | A | 1.41 | 350.1 |
| | 1-yl)-2-(6- | | | | | | | | | | | | |
| | (methylsulfonyl)-1H- | | | | | | | | | | | | |
| | indol-3-yl)ethane-1,2- | | | | | | | | | | | | |
| | dione | | | | | | | | | | | | |
| 83 | 3-[2-(4- | H | H | CN | H | H | H | NMe-piperazine | E | 10 | C | 2.84 | 297.1 |
| | methylpiperazin-1-yl)- | | | | | | | | | | | | |
| | 2-oxo-acetyl]-1H- | | | | | | | | | | | | |
| | indole-6-carbonitrile | | | | | | | | | | | | |
| 84 | 1-(6-methoxy-1H- | H | H | OMe | H | H | H | 3-methyl-3,8- | E | 14 | C | 3.17 | 328.1 |
| | indol-3-yl)-2-(3- | | | | | | | diazabicyclo[3.2.1] | | | | | |
| | methyl-3,8-diaza- | | | | | | | octane | | | | | |
| | bicyclo[3.2.1]oct-8-yl)- | | | | | | | | | | | | |
| | ethane-1,2-dione | | | | | | | | | | | | |
| 85 | 1-(6-methoxy-1H- | H | H | OMe | H | H | H | 8-methyl-3,8- | E | 3 | C | 3.20 | 328.1 |
| | indol-3-yl)-2-(8- | | | | | | | diazabicyclo[3.2.1] | | | | | |
| | methyl-3,8-diaza- | | | | | | | octane | | | | | |
| | bicyclo[3.2.1]oct-3-yl)- | | | | | | | | | | | | |
| | ethane-1,2-dione | | | | | | | | | | | | |

### Biological Experimental

Primary dorsal root ganglion neurons were isolated using standard techniques and placed in Ca²⁺/Mg²⁺ free phosphate-buffered saline (PBS). Hemisection of the spinal column and cord was performed and the ganglia were uncovered by gently lifting the spinal cord. They were placed in a dissociation solution (PBS) containing 1 mg/ml Collagenase (type XI) and trypsin (type II-S). Ganglia were incubated in the dissociation solution for 20 minutes at room temperature, followed by 20 minutes at 37° C. Ganglia were washed in culture medium, Dulbecco's Minimum essential medium (DMEM), supplemented with fetal calf serum (10%, Gibco), penicillin/strepromycin and glutamine (Glutamax, Gibco). Cells were dispersed by trituration and plated on poly-L-lysine-coated 35-mm dishes (BD Bioscience). Cells were kept in culture in a humidified atmosphere (37° C., 5% CO₂) for up to 4 days for electrophysiological recordings. Unless otherwise noted, all reagents were purchased from Sigma-Aldrich.

Barium currents carried by calcium channels were recorded using the whole-cell configuration of the patch-clamp technique. All experiments were carried out at room temperature. Cells were bathed in an external solution containing, in mM: TEA-CI, 140; BaCl₂, 5; MgCl₂, 1; HEPES, 10; pH 7.3 adjusted with TEA-OH. Ba²⁺ was used as a charge carrier to prevent the run-down caused by Ca²⁺-activated inactivation of Ca²⁺ channels. Patch pipettes had a resistance of 2-4 MΩ, when filled with a solution containing, in mM: CsCl, 140; EGTA, 5; MgCl₂, 2; HEPES, 10; pH adjusted to 7.3 with CsOH. Electrophysiological recordings were generated and acquired with a patch-clamp amplifier (Axopatch 200B, or Multiclamp 7; Molecular devices) connected to a personal computer. Online and offline analysis was carried out using the pClamp software suite (v.9, Molecular Devices). Compounds were all prepared from 100 mM stocks in 100% dimethyl sulfoxide (DMSO) and further diluted in external solution to achieve the desired final concentration. Final DMSO concentration was always <0.1%. For the assessment of activity of the compounds, only cells with a membrane capacitance (Cm)<40 pf were used, as the Ca2+ current in these cells is carried mostly through N-type/Cav2.2 channels. Cell membrane potential was held at −70 mV and currents were elicited by a series of 100-ms steps to 0 mV, at a frequency of 0.1 Hz. After the establishment of a steady baseline current, the compound was applied to the bath. When the block induced by the compound reached a steady level, the frequency was then increased to 0.5 Hz, to assess use-dependence. After stabilization of the high frequency block, step depolarizations were stopped and cell membrane potential was kept at −70 mV for 100-120 s, after which 0.1 Hz stimulation (100 ms, 0 mV) was elicited, to assess recovery of the block. ω-conotoxin GVIA (100 nM; Alomone labs) was added at the end of the experiment to quantify the remaining N-type current. Selectivity of the compounds over non-N-type currents was studied using similar stimulation protocols, in a bath solution containing ω-conotoxin GVIA (100 nM).

| | | Compound | % |
|---|---|---|---|
| Com- | | Concentration | Inhibition |
| pound | Name | (uM) | at 0.5 Hz |
| 20 | 2-(6-fluoro-1H-indol-3-yl)-2- | 1 | 55 |
| | oxoacetic acid | | |
| 31 | 2-(6-methoxy-1H-indol-3-yl)-2- | 0.1 | 36 |
| | oxoacetic acid | | |
| 40 | ethyl 2-(6-methoxy-1H-indol-3-yl)- | 1 | 57 |
| | 2-oxoacetate | | |
| 57 | 1-(6-methoxy-1H-indol-3-yl)-2- | 1 | 44 |
| | morpholinoethane-1,2-dione | | |
| 77 | 1-(6-methoxy-1H-indol-3-yl)-2-(4- | 1 | 55 |
| | methylpiperazin-1-yl)ethane-1,2- | | |
| | dione | | |

i Catterall, W. A., Perez-Reyes, E., Snutch, T. P., Striessnig, J. (2005) International Union of Pharmacology. XLVIII. Nomenclature and structure-function relationships of voltage-gated calcium channels. Pharmacol. Rev. 57, 411-425.
ii Berrow, N. S., Brice, N. L., Tedder, I., Page, K. M., Dolphin, A. C. (1997) Properties of cloned rat alpha1A calcium channels transiently expressed in the COS-7 cell line. Eur. J. Neurosci. 9, 739-748
iii Randall, A., Benham, C. D. (1999) Recent advances in the molecular understanding of voltage-gated Ca2+ channels. Mol. Cell. Neurosci. 14, 255-272.
iv Birnbaumer, L., Qin, N., Olcese, R., Tareilus, E., Platano, D., Costantin, J., Stefani, E. (1998) Structures and functions of calcium channel beta subunits. J. Bioenerg. Biomembr. 30, 357-375.
v Walker, D., De Waard, M. (1998) Subunit Interaction sites in voltage-dependent Ca2+ channels: role in channel function. Trends Neurosci. 21, 148-154.
vi Dolphin, A. C., Wyatt, C. N., Richards, J., Beattie, R. E., Craig, P., Lee, J. H., Cribbs, L. L., Volsen, S. G., Perez-Reyes, E. (1999) The effect of alpha2-delta and other accessory subunits on expression and properties of the calcium channel alpha1 G. J. Physiol. 519, Pt 1:35-45.
vii Lacerda, A. E., Perez-Reyes, E., Wei, X., Castellano, A., Brown, A. M. (1994) T-type and N-type calcium channels of Xenopus oocytes: evidence for specific Interactions with beta subunits. Biophys. J. 66, 1833-1843.
viii Dolphin, A. C. (2003) Beta subunits of voltage-gated calcium channels. J. Bioenerg. Biomembr. 35, 599-620.
ix Catterall, W. A., Perez-Reyes, E., Snutch, T. P., Striessnig, J. (2005) International Union of Pharmacology. XLVIII. Nomenclature and structure-function relationships of voltage-gated calcium channels. Pharmacol. Rev. 57, 411-425.
x Matthews, E. A., Dickenson, A. H. (2001) Effects of spinally delivered N- and P-type voltage-dependent calcium channel antagonists on dorsal horn neuronal responses in a rat model of neuropathy. Pain. 92, 235-246.
xi Dzhura, I., Neely, A. (2003) Differential modulation of cardiac Ca2+ channel gating by beta-subunits. Biophys. J. 85, 274-289.
xii Cahill, A. L., Hurley, J. H., Fox, A. P. (2000) Coexpression of cloned alpha(1B), beta(2a), and alpha(2)/delta subunits produces non-inactivating calcium currents similar to those found in bovine chromaffin cells. J. Neurosci. 20, 1685-1693.
xiii Scott, V. E., De Waard, M., Liu, H., Gurnett, C. A., Venzke, D. P., Lennon, V. A., Campbell, K. P. (1996) Beta subunit heterogeneity in N-type Ca2+ channels. J. Biol. Chem. 271, 3207-3212.
xiv Murakami, M., Felischmann, B., De Felipe, C., Freichel, M., Trost, C., Ludwig, A., Wissenbach, U., Schwegler, H., Hofmann, F., Hescheler, J., Flockerzi, V., Cavalie, A. (2002) Pain perception in mice lacking the beta3 subunit of voltage-gated calcium channels. J. Biol. Chem., 277, 40342-40351.
xv Birnbaumer, L., Qin, N., Olcese, R., Tareilus, E., Platano, D., Costantin, J., Stefani, E. (1998) Structures and functions of calcium channel beta subunits. J. Bioenerg. Biomembr. 30, 357-375.
xvi Canti, C., Bogdanov, Y., Dolphin, A. C. (2000) Interaction between G proteins and accessory subunits in the regulation of 1B calcium channels In Xenopus oocytes. J. Physiol. 527, Pt 3:419-432.
xvii Atanassoff, P. G., Hartmannsgruber, M. W., Thrasher, J., Wermeling, D., Longton, W., Gaeta, R., Singh, T., Mayo, M., McGuire, D., Luther, R. R. (2000). Ziconotide, a new N-type calcium channel blacker, administered intrathecally for acute postoperative pain. Reg. Anesth. Pain. Med. 25, 274-278.
xviii Dworkin, R. H., Portenoy, R. K. (1996) Pain and its persistence in herpes zoster. Pain. 67, 241-51.
xix Snutch, T. P., Sutton, K. G., Zamponi, G. W. (2001) Voltage-dependent calcium channels—beyond ihydropyridine antagonists. Curr. Opin. Pharmacol. 1, 11-16
xx Lyrica® Monograph. Micromedex Online. Located at http://www.micromedex.com. Accessed Sep. 23, 2005.
xxi Lyrica®. Electronic Orange Book. Available at http://www.fda.gov/cder/orange/default.htm. Accessed Sep. 25, 2005.
xxii Data on file. Pfizer Inc.; August 2005.
xxiii Carbone, A., Tubaro, A., Morello, P., Parascani, R., Catalan', C., Palleschi, G. (2003). The Effect Of Gabapentin On Neurogenic Detrusor Overactivity, A Pilot Study, Eur. Urol. 2(Suppl), 141, Abstract 555.
xxiv Yoshimura, N., Seki, S., de Groat, W. C. (2001) Nitrix oxide modulates Ca(2+) channels in dorsal root ganglion neurons innervating rat urinary bladder. J. Neurophysiol. 86, 304-311.
xxv Butcher, A. J., Leroy, J., Richards, M. W., Pratt, W. S., Dolphin, A. C. (2006) The Importance of occupancy rather than affinity of CaV(beta) subunits for the calcium channel I-II linker in relation to calcium channel function. J. Physiol. 574, 387-398.
xxvi Dalton, S., Takahashi, S. X., Miriyala, J., Colecraft, H. M. (2005) A single CaVbeta can reconstitute both trafficking and macroscopic conductance of voltage-dependent calcium channels. J. Physiol. 567, 757-769.
xxvii Arikkath, J., Campbell, K. P. (2003) Auxiliary subunits: essential components of the voltage-gated calcium channel complex. Curr. Opin. Neurobiol. 13, 298-307.
xxviii Yamaguchi, H., Nara, M., Strobeck, M., Fukasawa, K., Schwartz, A., Varadi, G. (1998) Multiple modulation pathways of calcium channel activity by a beta subunit. Direct evidence of beta subunit participation in membrane trafficking of the alpha1C subunit, J. Biol. Chem. 273, 19348-19356.
xxix Stotz, S. C., Barr, W., McRory, J. E., Chen, L., Jarvis, S. E., Zamponi, G. W. (2004) Several structural domains contribute to the regulation of N-type calcium channel inactivation by the beta 3 subunit. J. Biol. Chem. 279, 3793-3800.
xxx Stotz, S. C., Barr, W., McRory, J. E., Chen, L., Jarvis, S. E., Zamponi, G. W. (2004) Several structural domains contribute to the regulation of N-type calcium channel inactivation by the beta 3 subunit. J. Biol. Chem. 279, 3793-3800.

1-80. (canceled)

89. A compound represented by the general formula (I) or a pharmacologically acceptable salt or pro-drug thereof 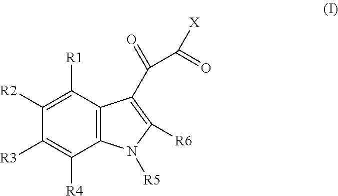
wherein:
R1, R2, R3 and R4 are independently selected from the group consisting of hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH2-O—;
R5 is a hydrogen atom or an alkyl group having from 1 to 6 carbon atoms;
R6 is a hydrogen atom or an alkyl group having from 1 to 6 carbon atoms; and
X is a group of formula NR8R9 wherein R8 and R9 together with the nitrogen atom to which they are attached form a 4- to 14-membered nitrogen-containing saturated or partially unsaturated heterocyclic group which has one or more rings (including bridged saturated or partially unsaturated heterocyclic groups having one or more rings), which further contains one or more additional nitrogen, oxygen or sulphur atoms, wherein said saturated or partially unsaturated heterocyclic group is optionally substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups,
provided that:
(i) when R8 and R9 together with the nitrogen atom to which they are attached form a piperazine group, and one or more of R1 to R4 are selected from the group consisting of hydrogen atoms, hydroxyl groups, nitro groups, amino groups, alkylamino groups, dialkylamino groups, alkoxycarbonylamino groups, halogen atoms, alkoxy groups and alkyl groups, the nitrogen atom of the piperazine group at the 4-position of the ring can not be substituted by an alkyl group,
(ii) when each of R1, R2, R3, R4, R5 and R6 is a hydrogen atom, then X can not be an unsubstituted piperazinyl group or an unsubstituted morpholino group, and
(iii) when each of R2 and R3 is a methoxy group or they together represent the moiety —O—CH2-O— and each of R1, R4, R5 and R6 is a hydrogen atom, then X can not be an unsubstituted morpholino group, or when R2 and R3 together represent the moiety —O—CH2-O— and each of R1, R4, R5 and R6 is a hydrogen atom, X cannot represent a 4-methylpiperazine group.

90. A compound according to claim 89 or a pharmacologically acceptable salt or prodrug thereof wherein R1, R2, R3 and R4 are independently selected from the group consisting of hydrogen atoms, methyl groups, ethyl groups, i-propyl groups, methoxy groups, ethoxy groups, trifluoromethyl groups, fluorine atoms, chlorine atoms, bromine atoms, trifluoromethoxy groups, hydroxymethyl groups, hydroxyl groups, cyano groups and methylsulphonyl groups or any two of R1 to R4 that are adjacent on the ring may together represent the moiety —O—CH2-O—.

91. A compound according to claim 89 or a pharmacologically acceptable salt or prodrug thereof wherein R5 is hydrogen or a methyl group.

92. A compound according to claim 89 or a pharmacologically acceptable salt or prodrug thereof wherein R6 is hydrogen or a methyl group.

93. A compound according to claim 89 or a pharmacologically acceptable salt or prodrug thereof wherein X is a group of formula NR8R9 wherein R8 and R9 together with the nitrogen atom to which they are attached form a 4- to 8-membered saturated or partially unsaturated nitrogen-containing heterocyclic group which has one or more rings (including bridged saturated or partially unsaturated heterocyclic groups having one or more rings), which further contains one or more additional nitrogen, oxygen or sulphur atoms, wherein said saturated or partially unsaturated heterocyclic group is optionally substituted by one or more substituents selected from the group consisting of alkyl groups having from 1 to 4 carbon atoms, halogen atoms, haloalkyl groups having from 1 to 4 carbon atoms, alkoxy groups having from 1 to 4 carbon atoms, alkoxycarbonyl groups comprising carbonyl groups that are substituted by alkoxy groups having from 1 to 4 carbon atoms, carboxyl groups, nitro groups, amino groups, monalkylamino groups wherein the alkyl groups have from 1 to 4 carbon atoms, dialkylamino groups wherein each alkyl group is the same or different and each is an alkyl group having from 1 to 4 carbon atoms and hydroxyl groups;
provided that:
(i) when R8 and R9 together with the nitrogen atom to which they are attached form a piperazine group, and one or more of R1 to R4 are selected from the group consisting of hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group, and
(iii) when each of R2 and R3 is a methoxy group or they together represent the moiety —O—CH2-O— and each of R1, R4, R5 and R6 is a hydrogen atom, then X can not be an unsubstituted morpholino group, or when R2 and R3 together represent the moiety —O—CH2-O— and each of R1, R4, R5 and R6 is a hydrogen atom X cannot represent a 4-methylpiperazine group.

94. A compound according to claim 89 or a pharmacologically acceptable salt or prodrug thereof wherein X is a group of formula NR8R9 wherein R8 and R9 together with the nitrogen atom to which they are attached form a 4- to 8-membered nitrogen-containing saturated heterocyclic group which has one or more rings (including bridged saturated or partially unsaturated heterocyclic groups having one or more rings), which further comprises an additional nitrogen, oxygen or sulphur atom, wherein said saturated heterocyclic group is optionally substituted by one or more substituents selected from hydroxyl groups, methyl groups and ethyl groups;
provided that:
(i) when R8 and R9 together with the nitrogen atom to which they are attached form a piperazine group, and one or more of R1 to R4 are selected from the group consisting of hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group, and
(iii) when each of R2 and R3 is a methoxy group or they together represent the moiety —O—CH2-O— and each of R1, R4, R5 and R6 is a hydrogen atom, then X can not be an unsubstituted morpholino group, or when R2 and R3 together represent the moiety —O—CH2-O— and each of R1, R4, R5 and R6 is a hydrogen atom X cannot represent a 4-methylpiperazine group.

95. A compound according to claim 89 or a pharmacologically acceptable salt or prodrug thereof wherein X is a group of formula NR8R9 wherein R8 and R9 together with the nitrogen atom to which they are attached form a morpholine ring, a piperazine ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring;
provided that when each of R1, R2, R3, R4, R5 and R6 is a hydrogen atom, then X can not be piperazine or morpholine.

96. A compound according to claim 89 or a pharmacologically acceptable salt or prodrug thereof wherein:
R1, R2, R3 and R4 are independently selected from the group consisting of hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH2-O—;
R5 is hydrogen or an alkyl group having from 1 to 6 carbon atoms;
R6 is hydrogen or an alkyl group having from 1 to 6 carbon atoms; and
X is a group of formula NR8R9 wherein R8 and R9 together with the nitrogen atom to which they are attached form a 4- to 8-membered nitrogen-containing saturated heterocyclic group having one or more rings (including bridged saturated or partially unsaturated heterocyclic groups having one or more rings), which further comprises an additional nitrogen, oxygen or sulphur atom, wherein said saturated heterocyclic group is optionally substituted by one or more substituents selected from hydroxyl groups, methyl groups and ethyl groups;
provided that:
(i) when R8 and R9 together with the nitrogen atom to which they are attached form a piperazine group, and one or more of R1 to R4 are selected from the group consisting of hydrogen atoms, hydroxyl groups, nitro groups, amino groups, alkylamino groups wherein the alkyl groups have from 1 to 4 carbon atoms, dialkylamino groups wherein each alkyl group is the same or different and each is an alkyl group having from 1 to 4 carbon atoms, alkoxycarbonylamino groups comprising a carbonylamino group which is substituted with an alkoxy group having from 1 to 4 carbon atoms, halogen atoms, alkoxy groups having from 1 to 4 carbon atoms and alkyl groups having from 1 to 4 carbon atoms, the nitrogen atom of the piperazine group at the 4-position of the ring can not be substituted by an alkyl group having from 1 to 6 carbon atoms,
(ii) when each of R1, R2, R3, R4, R5 and R6 is a hydrogen atom, then X can not be an unsubstituted piperazinyl group or an unsubstituted morpholino group, and
(iii) when each of R2 and R3 is a methoxy group or they together represent the moiety —O—CH2—O— and each of R1, R4, R5 and R6 is a hydrogen atom, then X can not be an unsubstituted morpholino group, or when R2 and R3 together represent the moiety —O—CH2—O— and each of R1, R4, R5 and R6 is a hydrogen atom X cannot represent a 4-methylpiperazine group.

97. A compound according to claim 89 or a pharmacologically acceptable salt or prodrug thereof wherein:
R1, R2, R3 and R4 are independently selected from the group consisting of hydrogen atoms, methyl groups, ethyl groups, i-propyl groups, methoxy groups, ethoxy groups, trifluoromethyl groups, fluorine atoms, chlorine atoms, bromine atoms, trifluoromethoxy groups, hydroxymethyl groups, hydroxyl groups, cyano groups and methylsulphonyl groups or any two of R1 to R4 that are adjacent on the ring may together represent the moiety —O—CH2—O—;
R5 is hydrogen or a methyl group;
R6 is hydrogen or a methyl group; and
X is a group of formula NR8R9 wherein R8 and R9 together with the nitrogen atom to which they are attached form a morpholine ring, a piperazine ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring;
provided that when each of R1, R2, R3, R4, R5 and R6 is a hydrogen atom, then X can not be piperazine or morpholine.

98. A compound of formula (I) or a pharmacologically acceptable salt or prodrug thereof according to claim 89 selected from the group consisting of:
1-(4-hydroxypiperidin-1-yl)-2-(6-methoxy-1H-indol-3-yl)-ethane-1,2-dione,
1-(5-fluoro-1H-indol-3-yl)-2-morpholinoethane-1,2-dione,
1-(6-fluoro-1H-indol-3-yl)-2-morpholinoethane-1,2-dione,
1-(7-fluoro-1H-indol-3-yl)-2-morpholinoethane-1,2-dione,
1-(6-chloro-1H-indol-3-yl)-2-morpholinoethane-1,2-dione,
1-(5-bromo-1H-indol-3-yl)-2-morpholinoethane-1,2-dione,
1-(1-methyl-1H-indol-3-yl)-2-morpholinoethane-1,2-dione,
1-(6-methyl-1H-indol-3-yl)-2-morpholinoethane-1,2-dione,
1-(2-methyl-1H-indol-3-yl)-2-morpholinoethane-1,2-dione,
1-(6-ethyl-1H-indol-3-yl)-2-morpholinoethane-1,2-dione,
1-(6-isopropyl-1H-indol-3-yl)-2-morpholinoethane-1,2-dione,
1-morpholino-2-(6-(trifluoromethyl)-1H-indol-3-yl)ethane-1,2-dione,
1-(5-methoxy-1H-indol-3-yl)-2-morpholinoethane-1,2-dione,
1-(6-methoxy-1H-indol-3-yl)-2-morpholinoethane-1,2-dione,
1-(6-ethoxy-1H-indol-3-yl)-2-morpholinoethane-1,2-dione,
1-(5,6-dimethoxy-1H-indol-3-yl)-2-morpholinoethane-1,2-dione,
1-(5H-[1,3]dioxolo[4,5-f]indol-7-yl)-2-morpholinoethane-1,2-dione,
1-morpholino-2-(6-(trifluoromethoxy)-1H-indol-3-yl)ethane-1,2-dione,
1-(6-(methylsulfonyl)-1H-indol-3-yl)-2-morpholinoethane-1,2-dione,
1-(4-methylpiperazin-1-yl)-2-(6-(trifluoromethoxy)-1H-indol-3-yl)ethane-1,2-dione,
1-(4-methylpiperazin-1-yl)-2-(6-(trifluoromethyl)-1H-indol-3-yl)ethane-1,2-dione,
1-(6-methoxy-1H-indol-3-yl)-2-piperazin-1-yl-ethane-1,2-dione,
1-(2-methyl-1H-indol-3-yl)-2-(4-methylpiperazin-1-yl)ethane-1,2-dione,
1-(5H-[1,3]dioxolo[4,5-f]indol-7-yl)-2-(4-methylpiperazin-1-yl)ethane-1,2-dione,
1-(4-methylpiperazin-1-yl)-2-(6-(methylsulfonyl)-1H-indol-3-yl)ethane-1,2-dione,
3-[2-(4-methylpiperazin-1-yl)-2-oxo-acetyl]-1H-indole-6-carbonitrile,
1-(6-methoxy-1H-indol-3-yl)-2-(3-methyl-3,8-diaza-bicyclo[3.2.1]oct-8-yl)-ethane-1,2-dione, and
1-(6-methoxy-1H-indol-3-yl)-2-(8-methyl-3,8-diaza-bicyclo[3.2.1]oct-3-yl)-ethane-1,2-dione.

99. An N-Me piperazine compound or a pharmacologically acceptable salt or prodrug thereof selected from the group consisting of:
1-(6-hydroxy-1H-indol-3-yl)-2-(4-methylpiperazin-1-yl)ethane-1,2-dione,
1-(4-fluoro-1H-indol-3-yl)-2-(4-methylpiperazin-1-yl)ethane-1,2-dione,
1-(5-fluoro-1H-indol-3-yl)-2-(4-methylpiperazin-1-yl)ethane-1,2-dione,
1-(6-fluoro-1H-indol-3-yl)-2-(4-methylpiperazin-1-yl)ethane-1,2-dione,
1-(7-fluoro-1H-indol-3-yl)-2-(4-methylpiperazin-1-yl)ethane-1,2-dione,
1-(6-chloro-1H-indol-3-yl)-2-(4-methylpiperazin-1-yl)ethane-1,2-dione,
1-(1-methyl-1H-indol-3-yl)-2-(4-methylpiperazin-1-yl)ethane-1,2-dione,
1-(6-methyl-1H-indol-3-yl)-2-(4-methylpiperazin-1-yl)ethane-1,2-dione,
1-(6-ethyl-1H-indol-3-yl)-2-(4-methylpiperazin-1-yl)ethane-1,2-dione,
1-(6-isopropyl-1H-indol-3-yl)-2-(4-methylpiperazin-1-yl)ethane-1,2-dione,
1-(5-methoxy-1H-indol-3-yl)-2-(4-methylpiperazin-1-yl)ethane-1,2-dione,
1-(6-methoxy-1H-indol-3-yl)-2-(4-methylpiperazin-1-yl)ethane-1,2-dione
1-(6-ethoxy-1H-indol-3-yl)-2-(4-methylpiperazin-1-yl)ethane-1,2-dione, and
1-(4-methylpiperazin-1-yl)-2-(6-(methylsulfonyl)-1H-indol-3-yl)ethane-1,2-dione.

100. A pharmaceutical composition comprising a pharmacologically acceptable diluent or carrier and an active ingredient, wherein said active ingredient is a compound of formula (I) according to claim 89 or an N-Me piperazine compound according to claim 99 or a pharmacologically acceptable salt or prodrug thereof, with the proviso that said composition does not contain 1-(1H-indol-3-yl)-2-morpholinoethane-1,2-dione.

101. A method for the prophylaxis or treatment of a disease or condition selected from the group consisting of inflammatory and immunological diseases, cell proliferative disorders, lower urinary tract disorders, anxiety and anxiety-related conditions, epilepsy, pain disorders, gynecological pain, cardiac arrhythmias, thromboembolic events, cardiovascular diseases, disorders of the auditory system, migraine, gastrointestinal disorders, vascular and visceral smooth muscle disorders, metabolic disorders, memory loss, CNS-mediated motor dysfunction disorders and ophthalmic disorders, comprising administering to a patient in need thereof a therapeutically effective amount of a compound of formula (II) or a pharmacologically acceptable salt or prodrug thereof 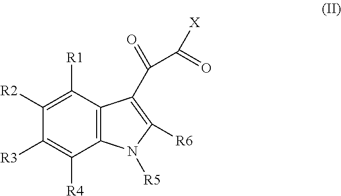
wherein:
R1, R2, R3 and R4 are independently selected from hydrogen atoms, alkyl groups having from 1 to 4 carbon atoms, alkoxy groups having from 1 to 4 carbon atoms, halogen atoms, hydroxyalkyl groups having from 1 to 4 carbon atoms, hydroxyl groups, haloalkyl groups having from 1 to 4 carbon atoms, haloalkoxy groups having from 1 to 4 carbon atoms, cyano groups and alkylsulfonyl groups having from 1 to 4 carbon atoms, or any two of R1 to R4 that are adjacent on the ring may together represent the moiety —O—CH2—O—;
R5 is a hydrogen atom or an alkyl group having from 1 to 6 carbon atoms;
R6 is a hydrogen atom or an alkyl group having from 1 to 6 carbon atoms; and
X is a group of formula NR8R9 wherein R8 and R9 together with the nitrogen atom to which they are attached form a 4- to 14-membered nitrogen-containing saturated or partially unsaturated heterocyclic group having one or more rings (including bridged saturated or partially unsaturated heterocyclic groups having one or more rings), which further contains one or more additional nitrogen, oxygen or sulphur atoms, said saturated or partially unsaturated heterocyclic group optionally being substituted by one or more substituents selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups that are substituted by alkoxy groups having from 1 to 6 carbon atoms, carboxyl groups, nitro groups, amino groups, monalkylamino groups wherein the alkyl groups have from 1 to 6 carbon atoms, dialkylamino groups wherein each alkyl group is the same or different and each is an alkyl group having from 1 to 6 carbon atoms and hydroxyl groups.

102. A method for the prophylaxis or treatment of a disease or condition according to claim 101, wherein the disease or condition is lower urinary tract disorders.

103. A method for the prophylaxis or treatment of a disease or condition according to claim 101, wherein the disease or condition is epilepsy.

104. A method for the prophylaxis or treatment of a disease or condition according to claim 101, wherein the disease or condition is pain disorders.

105. A method for the prophylaxis or treatment of a disease or condition according to claim 101, wherein the disease or condition is gynecological pain.

106. A method for the prophylaxis or treatment of a disease or condition according to claim 101, wherein the disease or condition is a migraine.

107. A method for the prophylaxis or treatment of a disease or condition according to claim 101, comprising administering a therapeutically effective amount of a compound of formula (II) or a pharmacologically acceptable salt or prodrug thereof, wherein X is a group of formula NR8R9 wherein R8 and R9 together with the nitrogen atom to which they are attached form a 4- to 8-membered saturated or partially unsaturated nitrogen-containing heterocyclic group which has one or more rings (including bridged saturated or partially unsaturated heterocyclic groups having one or more rings), which further contains one or more additional nitrogen, oxygen or sulphur atoms, wherein said saturated or partially unsaturated heterocyclic group is optionally substituted by one or more substituents selected from the group consisting of alkyl groups having from 1 to 4 carbon atoms, halogen atoms, haloalkyl groups having from 1 to 4 carbon atoms, alkoxy groups having from 1 to 4 carbon atoms, alkoxycarbonyl groups comprising carbonyl groups that are substituted by alkoxy groups having from 1 to 4 carbon atoms, carboxyl groups, nitro groups, amino groups, monalkylamino groups wherein the alkyl groups have from 1 to 4 carbon atoms, dialkylamino groups wherein each alkyl group is the same or different and each is an alkyl group having from 1 to 4 carbon atoms and hydroxyl groups.

108. A method for the prophylaxis or treatment of a disease or condition according to claim 101, comprising administering a therapeutically effective amount of a compound of formula (II) or a pharmacologically acceptable salt or prodrug thereof, wherein X is a group of formula NR8R9 wherein R8 and R9 together with the nitrogen atom to which they are attached form a 4- to 8-membered nitrogen-containing saturated heterocyclic group which has one or more rings (including bridged saturated or partially unsaturated heterocyclic groups having one or more rings), which further comprises an additional nitrogen, oxygen or sulphur atom, wherein said saturated heterocyclic group is optionally substituted by one or more substituents selected from hydroxyl groups, methyl groups and ethyl groups.

109. A method for the prophylaxis or treatment of a disease or condition according to claim 101, comprising administering a therapeutically effective amount of a compound of formula (II) or a pharmacologically acceptable salt or prodrug thereof, wherein X is a group of formula NR8R9 wherein R8 and R9 together with the nitrogen atom to which they are attached form a morpholine ring, a piperazine ring, a 4-methylpiperazin-1-yl ring, a N-methyl-3,8-diazabicyclo[3.2.1]octane ring, a 3-methyl-3,8-diaza-bicyclo[3.2.1]octane ring or a 8-methyl-3,8-diaza-bicyclo[3.2.1]octane ring.

110. A method for the prophylaxis or treatment of a disease or condition selected from a group consisting of inflammatory and immunological diseases, cell proliferative disorders, lower urinary tract disorders, anxiety and anxiety-related conditions, epilepsy, pain disorders, gynecological pain, cardiac arrhythmias, thromboembolic events, cardiovascular diseases, disorders of the auditory system, migraine, gastrointestinal disorders, vascular and visceral smooth muscle disorders, metabolic disorders, memory loss, CNS-mediated motor dysfunction disorders and ophthalmic disorders, the method comprising administering a therapeutically effective amount of a compound of formula (I) as defined in claim 98, an N-Me piperazine compound as defined in claim 99 or a compound selected from a group consisting of
2-(4-fluoro-1H-indol-3-yl)-2-oxoacetic acid,
2-(5-fluoro-1H-indol-3-yl)-2-oxoacetic acid,
2-(6-fluoro-1H-indol-3-yl)-2-oxoacetic acid,
2-(6-chloro-1H-indol-3-yl)-2-oxoacetic acid,
2-(5-bromo-1H-indol-3-yl)-2-oxoacetic acid,
2-(6-bromo-1H-indol-3-yl)-2-oxoacetic acid,
2-(1-methyl-1H-indol-3-yl)-2-oxoacetic acid,
2-(2-methyl-1H-indol-3-yl)-2-oxoacetic acid,
2-(5-methoxy-1H-indol-3-yl)-2-oxoacetic acid,
2-(6-methoxy-1H-indol-3-yl)-2-oxoacetic acid,
ethyl 2-(1H-indol-3-yl)-2-oxoacetate,
ethyl 2-(6-fluoro-1H-indol-3-yl)-2-oxoacetate,
ethyl 2-(6-methoxy-1H-indol-3-yl)-2-oxoacetate,
1-(5-bromo-1H-indol-3-yl)-2-(4-methylpiperazin-1-yl)ethane-1,2-dione,
1-(2-methyl-1H-indol-3-yl)-2-(4-methylpiperazin-1-yl)ethane-1,2-dione, and
1-(5,6-dimethoxy-1H-indol-3-yl)-2-(4-methylpiperazin-1-yl)ethane-1,2-dione,
or pharmacologically acceptable salts or prodrugs thereof.

111. A method for the prophylaxis or treatment of a disease or condition selected from the group consisting of lower urinary tract disorders, epilepsy, pain disorders, gynecological pain and migraine, the method comprising administering a therapeutically effective amount of an N-Me piperazine compound according to claim 99 or a pharmacologically acceptable salt or prodrug thereof to a patient in need thereof.

112. A pharmaceutical composition comprising a compound of claim 89 or a compound of claim 99 in combination with a pharmacologically acceptable diluent, carrier or excipient.

113. A pharmaceutical composition comprising a pharmacologically acceptable diluent or carrier; at least two compounds selected from a group comprising:
a compound of formula (I) as defined in claim 89 or a pharmacologically acceptable salt or prodrug thereof,
an N-methyl piperazine derivative as defined in claim 99 or a pharmacologically acceptable salt or prodrug thereof, or
a compound of formula (II) as defined in claim 101 or a pharmacologically acceptable salt or prodrug thereof; and
at least one compound selected from the group consisting of muscarinic receptor antagonists, P3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists, alpha-1 adrenoceptor antagonists, tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

114. A pharmaceutical composition comprising a pharmacologically acceptable diluent or carrier; at least one of:
a compound of formula (I) as defined in claim 89 or a pharmacologically acceptable salt or prodrug thereof,
an N-methyl piperazine derivative as defined in claim 99 or a pharmacologically acceptable salt or prodrug thereof, or
a compound of formula (II) as defined in claim 101 or a pharmacologically acceptable salt or prodrug thereof; and
at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists and α-1 adrenoceptor antagonists.

115. A pharmaceutical composition comprising a pharmacologically acceptable diluent or carrier; at least one of:
a compound of formula (I) as defined in claim 89 or a pharmacologically acceptable salt or prodrug thereof,
an N-methyl piperazine derivative as defined in claim 99 or a pharmacologically acceptable salt or prodrug thereof,
or a compound of formula (II) as defined in claim 101 or a pharmacologically acceptable salt or prodrug thereof; and
at least one compound selected from the group consisting of neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

116. A method for the prophylaxis or treatment of lower urinary tract disorders comprising administering a therapeutically effective amount of at least one of:
a compound of formula (I) according to claim 89 or a pharmacologically acceptable salt or prodrug thereof,
an N-methyl piperazine derivative as defined in claim 99 or a pharmacologically acceptable salt or prodrug thereof, or
a compound of formula (II) as defined in claim 101 or a pharmacologically acceptable salt or prodrug thereof; and
at least one compound selected from the group consisting of muscarinic receptor antagonists, β3 adrenergic receptor agonists, neurokinin K receptor antagonists, vanilloid VR1 agonists, calcium channel α2 δ delta ligands, potassium channel inhibitors, calcium channel inhibitors, sodium channel blockers, serotonin and norepinephrine reuptake inhibitors (SNRIs), 5-HT antagonists and α-1 adrenoceptor antagonists.

117. A method for the prophylaxis or treatment of pain comprising administering a therapeutically effective amount of at least one of:
a compound of formula (I) as defined in claim 89 or a pharmacologically acceptable salt or prodrug thereof,
an N-methyl piperazine derivative as defined in claim 99 or a pharmacologically acceptable salt or prodrug thereof,
or a compound of formula (II) as defined in claim 101 or a pharmacologically acceptable salt or prodrug thereof; and
at least one compound selected from the group consisting of neurokinin K receptor antagonists, vanilloid VR1 agonists, calcium channel α2 δ delta ligands, potassium channel inhibitors, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs) in the manufacture of a medicament for the prophylaxis or treatment of pain.

118. A method for the prophylaxis or treatment of a disease or condition selected from the group consisting of inflammatory and immunological diseases, cell proliferative disorders, lower urinary tract disorders, anxiety and anxiety-related conditions, epilepsy, pain disorders, gynecological pain, cardiac arrhythmias, thromboembolic events, cardiovascular diseases, disorders of the auditory system, migraine, gastrointestinal disorders, vascular and visceral smooth muscle disorders, metabolic disorders, memory loss, CNS-mediated motor dysfunction disorders and ophthalmic disorders, the method comprising administering to a patient in need thereof a therapeutically effective amount of a pharmaceutical composition comprising a pharmacologically acceptable diluent or carrier; at least one of:
a compound of formula (I) as defined in claim 89 or a pharmacologically acceptable salt or prodrug thereof,
an N-methyl piperazine derivative as defined in claim 99 or a pharmacologically acceptable salt or prodrug thereof,
or a compound of formula (II) as defined in claim 101 or a pharmacologically acceptable salt or prodrug thereof; and
at least one compound selected from the group consisting of neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands non-steroidal anti-inflammatory drugs (NSAIDs), muscarinic receptor antagonists, β3 adrenergic receptor agonists, 5-HT antagonists and α-1 adrenoceptor antagonists.
